(19) United States
(12) Patent Application Publication
(10) Pub. No.: US 2019/0185556 A1
(43) Pub. Date: Jun. 20, 2019

(54) METHOD OF IMPROVING CONNECTIVE TISSUE ATTACHMENT USING ANTI-SCLEROSTIN ANTIBODIES

(71) Applicants: AMGEN INC., Thousand Oaks, CA (US); WASHINGTON UNIVERSITY, St. Louis, MO (US)

(72) Inventors: Michael S. Ominsky, Thousand Oaks, CA (US); Stavros Thomopoulos, New York, NY (US)

(21) Appl. No.: 16/323,470

(22) PCT Filed: Aug. 7, 2017
(86) PCT No.: PCT/US17/45705

§ 371 Date: Feb. 5, 2019

Related U.S. Application Data
(60) Provisional application No. 62/372,124, filed on Aug. 8, 2016.

(51) Int. Cl. C07K 16/22; A61P 41/00; A61L 27/22; A61L 27/56

(52) U.S. Cl. CPC C07K 16/22; A61P 41/00; A61K 38/00; A61L 27/56; A61L 27/22

(57) ABSTRACT

The application provides method for enhancing connective tissue-to-bone healing in a subject in need thereof comprising administering to the subject an anti-sclerostin antibody in an amount effective to enhance connective tissue-to-bone healing in the subject.

### INCORPORATION BY REFERENCE

The following applications are hereby incorporated by reference in their entirety: International Patent Application No. PCT/US2012/049331, filed Aug. 2, 2012, which claims priority to U.S. Provisional Patent Application No. 61/515,191, filed Aug. 4, 2011; U.S. patent application Ser. No. 11/410,540, filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005; and U.S. patent application Ser. No. 11/411,003 (issued as U.S. Pat. No. 7,592,429), filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005. The following applications also are hereby incorporated by reference: U.S. patent application Ser. No. 12/212,327, filed Sep. 17, 2008, which claims priority to U.S. Provisional Patent Application No. 60/973,024, filed Sep. 17, 2007; and U.S. patent application Ser. No. 12/811,171, filed Jun. 29, 2010, which is a U.S. National Phase Application pursuant to 35 U.S.C. § 371 of International Patent Application No. PCT/US08/86864, filed on Dec. 15, 2008, which claims priority to U.S. Provisional Patent Application No. 61/013,917, filed Dec. 14, 2007.

### STATEMENT OF GOVERNMENT SUPPORT

This invention was made with government support under Grant No. F31-AR066452 and No. R01-AR057836 awarded by the National Institutes of Health (NIH). The government has certain rights in the invention.

### FIELD OF THE INVENTION

The present disclosure is directed to the use of anti-sclerostin antibodies to enhance connective tissue-to-bone healing.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ASCII (text) file named “50928A_SeqListing.txt,” 806,135 bytes, created on Aug. 7, 2017.

### BACKGROUND

Rotator cuff tears are one of the most common injuries to the upper extremity; the incidence of full-thickness tears is approximately 25% in the population over the age of 60 and 50% in the population over the age of 80 [1, 2]. Tears are debilitating and do not heal spontaneously, typically becoming larger within a few years after injury [3]. This leads to over 250,000 rotator cuff surgical repairs in the United States annually. Unfortunately, poor tendon-to-bone healing after repair results in an alarmingly high incidence of re-tears, ranging from 20% in young healthy patients with small tears to 94% in older patients with massive tears [4, 5]. Poor healing is characterized by loss of bone at the healing interface and a lack of regeneration of the functionally graded mineralized fibrocartilage found in the healthy attachment [6]. Accordingly, there exists a need for treatments and therapies to improve tendon-to-bone healing. The present invention meets this need and provides related advantages.

### SUMMARY

In one aspect, described herein is a method for enhancing connective tissue-to-bone healing in a subject in need thereof comprising administering to the subject an anti-sclerostin antibody in an amount effective to enhance connective tissue-to-bone healing in the subject. Exemplary connective tissues include, but are not limited to, a ligament, a tendon, a meniscus or a labrum.

In some or any embodiments, the anti-sclerostin antibody is administered along with a second bone-enhancing therapeutic for the treatment of decreased bone mineral density or bone fracture. Many therapeutics of this type are known in the art. In some embodiments, the bone-enhancing therapeutic is selected from the group consisting of an anti-resorptive drug, a bone-forming agent, an estrogen receptor antagonist (including, but not limited to, raloxifene, bazedoxifene and lasofoxifene) and a drug that has an inhibitory effect on osteoclasts. In some embodiments, the anti-resorptive drug includes, but is not limited to, parathyroid hormone, a bisphosphonate (including, but not limited to, alendronate, risedronate, ibandronate and zoledronate), an estrogen or estrogen analogue, a selective estrogen receptor modulator (SERM) and a calcium source, Tibolone, calcitonin, a calcitriol and hormone replacement therapy. In some embodiments, the bone-enhancing agent includes, but is not limited to parathyroid hormone (PTH) or a peptide fragment thereof, PTH-related protein (PTHrp), bone morphogenetic protein, osteogenin, NaF, a PGE2 agonist, a statin, an anti-DKK1 antibody or inhibitor, an anti-RANK ligand (RANKL) antibody or RANKL inhibitor, strontium ranelate, vitamin D, or a vitamin D derivative or mimic thereof. In some embodiments, the bone-enhancing agent is Forteo® (Teriparatide, or recombinant human parathyroid hormone 1-34) or Preotact® (parathyroid hormone). In some or any embodiments, the bone-enhancing agent is Protelos®.

The use of an anti-sclerostin antibody disclosed in U.S. Patent Publication No. 20070110747 (the disclosure of which is incorporated herein by reference in its entirety) in any of the methods disclosed herein or for preparation of medicaments for administration according to any of the methods disclosed herein, is specifically contemplated. One or more doses of the anti-sclerostin antibody are administered in an amount and for a time effective to enhance connective tissue-to-bone healing or to improve the outcome of a connective tissue reattachment procedure in a subject. One or more doses of anti-sclerostin antibody can comprise between about 70 mg to about 300 mg. or about 90 mg to about 270 mg. For example, the dose of anti-sclerostin antibody may range from at least about 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg or 300 mg. Ranges between any and all of these endpoints are also contemplated, e.g. about 90 mg to about 270 mg, about 70 mg to about 210 mg, about 100 mg to about 210 mg, about 90 mg to about 250 mg, about 110 mg to about 210 mg, about 70 mg to about 300 mg, or about 175 to about 270 mg.

Also described herein is the use of an effective amount of an anti-sclerostin antibody for improving the outcome of a connective tissue reattachment procedure in a mammalian subject in need thereof. Exemplary connective tissue reattachment procedures include, but are not limited to, rotator cuff repair, Achilles tendon repair, patellar-patella tendon repair, medial cruciate ligament (MCL) reconstruction, anterior cruciate ligament (ACL) reconstruction, ulnar collateral ligament (UCL), meniscus repair and labrum repair.

In some embodiments, the procedure comprises graft attachment, and the anti-sclerostin antibody is applied to the graft ex vivo.

In any of the methods or uses described herein, in some embodiments, the anti-sclerostin antibody is administered systemically (e.g., by subcutaneous injection. In other embodiments, the anti-sclerostin antibody is incorporated into a gel, a sponge, or matrix and implanted locally.

In some embodiments, the anti-sclerostin antibody for use in the methods described herein binds to sclerostin of SEQ ID NO: 1, with an affinity (Kd) of less than or equal to 1×10 7 M (or less than or equal to 1×10 8 M, or less than or equal to 1×10⁹ M, or less than or equal to 1×10¹⁰ M, or less than or equal to 1×10¹¹ M, or less than or equal to 1×10¹² M).

In various embodiments, the anti-sclerostin antibody binds to a sclerostin polypeptide comprising the amino acid sequence set forth in SEQ ID NO: 1 and binds the sequence of SEQ ID NO: 6 (CGPARLLPNAIGRGKWWRPSGPDFRC; corresponding to amino acids 86-111 of SEQ ID NO: 1). Alternatively or in addition, the anti-sclerostin antibody binds to a sclerostin polypeptide comprising the amino acid sequence set forth in SEQ ID NO: 1 and binds the sequence of at least one of SEQ ID NO: 2 (DVSEYSCRELHFTR; corresponding to amino acids 51-64 of SEQ ID NO: 1), SEQ ID NO: 3 (SAKPVTELVCSGQCGPAR; corresponding to amino acids 73-90 of SEQ ID NO: 1), SEQ ID NO: 4 (WWRPSGPDFRCIPDRYR; corresponding to amino acids 101-117 of SEQ ID NO: 1), SEQ ID NO: 5 (LVASCKCKRLTR; corresponding to amino acids 138-149 of SEQ ID NO: 1), SEQ ID NO: 70 (SAKPVTELVCSGQC; corresponding to amino acids 73-86 of SEQ ID NO: 1), SEQ ID NO: 71 (LVASCKC; corresponding to amino acids 138-144 of SEQ ID NO: 1), SEQ ID NO: 72 (CRELHFTR; corresponding to amino acids 57-64 of SEQ ID NO: 1), or SEQ ID NO: 73 (CIPDRYR; corresponding to amino acids 111-117 of SEQ ID NO: 1) within SEQ ID NO: 1. For example, in one aspect, the anti-sclerostin antibody binds a subregion of sclerostin of SEQ ID NO: 1 comprising SEQ ID NOs: 2-5 (and/or SEQ ID NOs: 70-73), optionally in its native three-dimensional conformation. Optionally, the anti-sclerostin antibody binds a peptide consisting of one or more of SEQ ID NO: 2, SEQ ID NO: 3, SEQ ID NO: 4, SEQ ID NO: 5, SEQ ID NO: 6, SEQ ID NO: 70, SEQ ID NO: 71, SEQ ID NO: 72, or SEQ ID NO: 73 (e.g., a peptide consisting of SEQ ID NO: 2, SEQ ID NO: 3, SEQ ID NO: 4, and SEQ ID NO: 5 or a peptide consisting of SEQ ID NO: 70, SEQ ID NO: 71, SEQ ID NO: 72, and SEQ ID NO: 73).

In various aspects, the anti-sclerostin antibody is capable of neutralizing human sclerostin in a MC3T3 cell-based mineralization assay when there is less than a 6-fold excess of moles of sclerostin binding sites per well as compared to the number of moles of sclerostin per well.

The anti-sclerostin antibody optionally has an IC50 of 100 nM or less, or 75 nM or less, or 50 nM or less, or 25 nM or less for neutralizing human sclerostin in a cell-based assay, such as a bone specific alkaline phosphatase assay. Alternatively or in addition, the anti-sclerostin antibody has an IC50 of 100 nM or less (e.g., 75 nM or less, or 50 nM or less) for neutralizing human sclerostin in a cell-based Wnt signaling assay in HEK293 cell lines, such as the Wnt assay involving Wntl-mediated induction of STF reporter gene. Alternatively or in addition, the anti-sclerostin antibody has an IC50 of 500 nM or less (e.g., 250 nM or less, 150 nM or less, 100 nM or less, or 50 nM or less) for neutralizing human sclerostin in a BMP2-induced mineralization assay in MC3T3 cells.

In one embodiment, the anti-sclerostin antibody cross-blocks the binding of at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24 to sclerostin and/or is cross-blocked from binding to sclerostin by at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24.

In some embodiments, the anti-sclerostin antibody comprises a CDR-H1 of SEQ ID NO:245, a CDR-H2 of SEQ ID NO:246, a CDR-H3 of SEQ ID NO:247, a CDR-L1 of SEQ ID NO:78, a CDR-L2 of SEQ ID NO:79 and a CDR-L3 of SEQ ID NO:80.

In one embodiment, the anti-sclerostin antibody comprises heavy chains comprising SEQ ID NO: 378 and light chains comprising SEQ ID NO: 376. In another embodiment, anti-sclerostin antibody has heavy chains of SEQ ID NO: 145 or SEQ ID NO: 392 and light chains of SEQ ID NO: 141.

In another embodiment, the anti-sclerostin antibody comprises CDRs of SEQ ID NOs: 20-25 of International Patent Publication No. WO 2008/115732 (SEQ ID NOs: 416-421), CDRs of SEQ ID NOs: 26-31 of International Patent Publication No. WO 2008/115732 (SEQ ID NOs: 422-427), CDRs of SEQ ID NOs: 32-37 of International Patent Publication No. WO 2008/115732 (SEQ ID NOs: 428-433), or CDRs of SEQ ID NOs: 4, 15, 26, 37, 48, and 59 of International Patent Publication No. WO 2009/047356 (SEQ ID NOs: 443, 454, 465, 476, 487, and 498, respectively). In yet another embodiment, the anti-sclerostin antibody comprises an amino acid sequence of at least one of SEQ ID NOs: 135-143, 153-161, or 171-179 of International Patent Publication No. WO 2010/130830 (SEQ ID NOs: 745-753, 763-771, 781-789, respectively).

In some embodiments, the anti-sclerostin antibody is formulated into a pharmaceutical composition comprising 55 mM acetate, 13 mm calcium, 6.0% (w/v) sucrose, 0.006% (w/v) polysorbate 20, at pH 5.2. In some embodiments, the pharmaceutical composition comprises 90 mg/mL anti-sclerostin antibody.

The section headings used herein are for organizational purposes only and are not to be construed as limiting the subject matter described. All references cited within the body of this specification are expressly incorporated by reference in their entirety.

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3rd ed., Cold Spring Harbor Laboratory Press, cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well known and commonly used in the art. Standard techniques may be used for chemical synthesis, chemical analyses, pharmaceutical preparation, formulation, and delivery and treatment of patients.

### BRIEF DESCRIPTION OF THE FIGURES

FIGS. 1A-1D show that treatment with Scl-Ab in the rotator cuff animal model increased bone mass indices (FIG. 1A) bone volume per total volume (BV/TV), (FIG. 1B) bone mineral density (BMD), (FIG. 1C) trabecular number (TbN), and (FIG. 1D) trabecular thickness (TbTh) in the region around the tendon-to-bone insertion site in the normal (non-injured) and 8 week healing groups. Significant effect of Scl-Ab is indicated by a line over bars (p<0.05; ANOVA followed by Tukey's post hoc compared to CTL within Group). Significant difference compared to normal is indicated by an “a” within a bar (p<0.05; ANOVA followed by Tukey's post hoc compared to normal within a particular treatment group).

FIGS. 2A-2D show that treatment with Scl-Ab in the rotator cuff animal model led to increased attachment site (FIG. 2A) failure load, (FIG. 2B) strength, and (FIG. 2C) stiffness after 8 weeks of healing, with failure load and stiffness returning to levels that were similar to normal (non-injured) attachments relative to controls. Stiffness and (FIG. 2D) Modulus were decreased in Scl-Ab treated normal (non-injured) attachments. Significant effect of Scl-Ab is indicated by a line over bars (p<0.05; ANOVA followed by Tukey's post hoc compared to CTL within Group). Significant difference compared to normal is indicated by an “a” within a bar (p<0.05; ANOVA followed by Tukey's post hoc compared to normal within a particular treatment group).

FIGS. 3A-3D show that after 8 weeks of healing, Scl-Ab treatment improved insertion continuity, integrity, and fiber alignment (FIGS. 3B and 3D) compared to CTL (FIGS. 3A and 3C). The enthesis area is outlined with a white dashed box, and magnified in FIG. 3C and FIG. 3D. Scale bars=1 mm for FIGS. 3A and 3B; scale bars=250 μm for FIGS. 3C and 3D.

FIG. 4A shows the gene expression of sclerostin, Dkk1, Lrp5, OCN, Pth1r, RankL, OPG, DMP1, Osterix, Runx2, Ctsk and Col2a1 in mineralized tissue adjacent to the tendon enthesis relative to the housekeeping gene RPL13a. FIG. 4B shows the gene expression of Acan, TFGβ1, TGβ3, MMP2, Sox9, Smo and Notch1 in mineralized tissue adjacent to the tendon enthesis relative to the housekeeping gene RPL13a. Significant effect of Scl-Ab is indicated by a line over bars (p<0.05; ANOVA followed by Tukey's post hoc compared to CTL within Group). Significant difference compared to normal is indicated by an “a” within a bar (p<0.05; ANOVA followed by Tukey's post hoc compared to normal within a particular treatment group). Significant effect of Scl-Ab compared to Normal in CTL group is indicated by a “b” within a bar (p<0.05, ANOVA followed by Tukey's post hoc).

FIG. 5A shows the gene expression of Sderaxis, Tenomodulin, Col1a1, Aggrecan, MMP2 and Smp in the tendon relative to the housekeeping gene RPL13a. FIG. 5B shows the gene expression of Col1a2, Col2a1, Col3a1, Sox9, TGβ1, TGβ3 and Notch1 in the tendon relative to the housekeeping gene RPL13a. Significant difference compared to normal is indicated by an “a” within a bar (p<0.05; ANOVA followed by Tukey's post hoc compared to normal within a particular treatment group).

FIG. 6 is a chart listing amino acid sequences and sequence identifiers for amino acid sequences of various anti-sclerostin antibodies described herein. The sequence identifiers refer to amino acid sequences provided in the Sequence Listing submitted herewith. The amino acid sequences also are set forth in U.S. Patent Publication No. 2007/0110747 or International Patent Publication Nos. WO 2008/115732, WO2009/047356, or WO 2010/130830, hereby incorporated by reference.

### DETAILED DESCRIPTION

Rotator cuff tears are common and lead to pain and disability. Poor healing after surgical repair, including significant loss of bone at the interface, leads to a high rate of re-tear. As described in the Examples, treatment with an anti-sclerostin antibody prevents bone loss and enhances rotator cuff healing in an animal model. As demonstrated herein, after 8 weeks of healing, animals receiving anti-sclerostin antibody (Scl-Ab) treatment had 30% greater bone mineral density than matched controls. A decrease in biomechanical properties was observed in both groups after 2 and 4 weeks of healing compared to healthy tendon-to-bone attachments. After 8 weeks of healing, Scl-Ab treated animals had improved strength (38%) and stiffness (43%) compared to control animals. Histological assessment showed that Scl-Ab promoted better integration of tendon and bone by 8 weeks of healing. Scl-Ab also had significant effects on osteoblast, osteoclast, and osteoprogenitor gene expression in bone, indicating enhanced bone formation. Scl-Ab treatment had no effect on expression of genes in tendon.

In one aspect, described herein is a method for enhancing connective tissue-to-bone healing in a subject in need thereof comprising administering to the subject an anti-sclerostin antibody in amount effective to enhance connective tissue-to-bone healing in the subject. In some embodiments, the connective tissue is a ligament, tendon, meniscus or labrum. In other embodiments, the connective tissue is a tendon. In still further embodiments, the connective tissue is ligament and tendon. The phrase “enhancing connective tissue-to-bone healing” as used herein refers to an earlier, stronger attachment between connective tissue and bone.

Antibodies

The term “antibody” refers to an intact antibody. An antibody may comprise a complete antibody (immunoglobulin) molecule (including polyclonal, monoclonal, chimeric, humanized, and/or human versions having full length heavy and/or light chains).

The term “antibody fragment” as used herein refers to an antigen-binding portion of an antibody. Antibody fragments include F(ab′)₂, Fab, Fab′, Fv, Fc, and Fd fragments, and can be incorporated into single domain antibodies (e.g., nanobodies), single-chain antibodies, maxibodies, minibodies, intrabodies, diabodies, triabodies, tetrabodies, v-NAR and bis-scFv (see, e.g., Hollinger and Hudson, Nature Biotechnology, 23(9):1126-1136 (2005)). Antibody polypeptides, including fibronectin polypeptide monobodies, also are disclosed in U.S. Pat. No. 6,703,199. Other antibody polypeptides are disclosed in U.S. Patent Publication No. 20050238646. The methods and antibody chains described herein are useful for generating heterodimeric antibodies, as described in, for example U.S. Patent Application Publication Nos. US 2014/154254, the disclosure of which is incorporated herein by reference in its entirety. The features of antibodies described herein, as well as discussion of timing and route of administration, also apply to antibody fragments.

An antibody fragment may be a synthetic or genetically engineered protein. For example, antibody fragments include isolated fragments consisting of the light chain variable region, “Fv” fragments consisting of the variable regions of the heavy and light chains, and recombinant single chain polypeptide molecules in which light and heavy variable regions are connected by a peptide linker (scFv proteins).

Another form of an antibody fragment is a peptide comprising one or more complementarity determining regions (CDRs) of an antibody. As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDRs (also termed “minimal recognition units” or “hypervariable region”) are obtained by, e.g., constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

An “anti-sclerostin antibody” binds to sclerostin or portions thereof to block or impair binding of human sclerostin to one or more ligands. Sclerostin, the product of the SOST gene, is absent in sclerosteosis, a skeletal disease characterized by bone overgrowth and strong dense bones (Brunkow et al., Am. J. Hum. Genet., 68:577-589 (2001); Balemans et al., Hum. Mol. Genet., 10:537-543 (2001)). The amino acid sequence of human sclerostin is reported by Brunkow et al. and is disclosed in U.S. Patent Publication No. 20070110747 as SEQ ID NO: 1 (which patent publication is incorporated in its entirety for its description of sclerostin binding agents and Sequence Listing). Recombinant human sclerostin/SOST is commercially available from R&D Systems (Minneapolis, Minn., USA; 2006 Catalog #1406-ST-025). Additionally, recombinant mouse sclerostin/SOST is commercially available from R&D Systems (Minneapolis, Minn., USA; 2006 Catalog #1589-ST-025). Research grade sclerostin-binding monoclonal antibodies are commercially available from R&D Systems (Minneapolis, Minn., USA; mouse monoclonal: 2006 Catalog # MAB1406; rat monoclonal: 2006 Catalog # MAB1589). U.S. Pat. Nos. 6,395,511 and 6,803,453, and U.S. Patent Publication Nos. 2004/0009535 and 2005/0106683 refer to anti-sclerostin antibodies generally. Examples of sclerostin antibodies or fragments thereof suitable for use in the context of the invention also are described in U.S. Patent Publication Nos. 2007/0110747 and 2007/0072797, which are hereby incorporated by reference. Additional information regarding materials and methods for generating sclerostin binding agents can be found in U.S. Patent Publication No. 20040158045 (hereby incorporated by reference).

Anti-sclerostin antibodies or fragments thereof may bind to sclerostin of SEQ ID NO: 1, or a naturally occurring variant thereof, with an affinity (Kd) of less than or equal to 1×10⁻⁷ M, less than or equal to 1×10⁻⁸M, less than or equal to 1×10⁻⁹ M, less than or equal to 1×10⁻¹⁰ M, less than or equal to 1×10⁻¹¹ M, or less than or equal to 1×10⁻¹² M. For example, the anti-sclerostin antibody binds sclerostin with a binding affinity of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10 M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. “Specifically binds” as used herein means that the antibody or fragment thereof binds sclerostin over other proteins. In some embodiments “specifically binds” means the antibody or fragment thereof has a higher affinity for sclerostin than for other proteins. Affinity is determined using a variety of techniques, an example of which is an affinity ELISA assay. In various embodiments, affinity is determined by a BIAcore assay. In various embodiments, affinity is determined by a kinetic method. In various embodiments, affinity is determined by an equilibrium/solution method. U.S. Patent Publication No. 2007/0110747 contains additional description of affinity assays suitable for determining the affinity (Kd) of an antibody for sclerostin. Exemplary affinity assays are described in Examples 10 and 11 of U.S. Patent Publication No. 2008/0110747, the disclosure of which is incorporated by reference in its entirety.

In some or any embodiments, the anti-sclerostin antibody or antibody fragment binds to a sclerostin polypeptide comprising the amino acid sequence set forth in SEQ ID NO: 1 and binds a region of sclerostin comprising the sequence of SEQ ID NO: 6 (CGPARLLPNAIGRGKWWRPSGPDFRC; corresponding to amino acids 86-111 of SEQ ID NO: 1). This region is also referred to herein as the “loop 2” region of sclerostin. Regions of sclerostin outside of the loop 2 region are defined herein as “non-loop 2 regions.” Alternatively or in addition, the anti-sclerostin antibody binds to a sclerostin polypeptide comprising amino acids 57-146 of SEQ ID NO: 1. Alternatively or in addition, the anti-sclerostin antibody binds to a sclerostin polypeptide comprising amino acids 89-103 of SEQ ID NO: 1 and/or amino acids 137-151 of SEQ ID NO: 1. Alternatively or in addition, the anti-sclerostin antibody binds to a sclerostin polypeptide comprising the amino acid sequence set forth in SEQ ID NO: 1 and binds the sequence of at least one of SEQ ID NO: 2 (DVSEYSCRELHFTR; corresponding to amino acids 51-64 of SEQ ID NO: 1), SEQ ID NO: 3 (SAKPVTELVCSGQCGPAR; corresponding to amino acids 73-90 of SEQ ID NO: 1), SEQ ID NO: 4 (WWRPSGPDFRCIPDRYR; corresponding to amino acids 101-117 of SEQ ID NO: 1), SEQ ID NO: 5 (LVASCKCKRLTR; corresponding to amino acids 138-149 of SEQ ID NO: 1), SEQ ID NO: 70 (SAKPVTELVCSGQC; corresponding to amino acids 73-86 of SEQ ID NO: 1), SEQ ID NO: 71 (LVASCKC; corresponding to amino acids 138-144 of SEQ ID NO: 1), SEQ ID NO: 72 (C1RELHFTR; corresponding to amino acids 57-64 of SEQ ID NO: 1), or SEQ ID NO: 73 (CIPDRYR; corresponding to amino acids 111-117 of SEQ ID NO: 1) within SEQ ID NO: 1. For example, in one aspect, the anti-sclerostin antibody binds a subregion of sclerostin of SEQ ID NO: 1 comprising SEQ ID NOs: 2-5 (and/or SEQ ID NOs: 70-73), optionally in its native three-dimensional conformation. Optionally, the anti-sclerostin antibody binds a peptide consisting of one or more of SEQ ID NO: 2, SEQ ID NO: 3, SEQ ID NO: 4, SEQ ID NO: 5, SEQ ID NO: 6, SEQ ID NO: 70, SEQ ID NO: 71, SEQ ID NO: 72, or SEQ ID NO: 73 (e.g., a peptide consisting of SEQ ID NO: 2, SEQ ID NO: 3, SEQ ID NO: 4, and SEQ ID NO: 5 or a peptide consisting of SEQ ID NO: 70, SEQ ID NO: 71, SEQ ID NO: 72, and SEQ ID NO: 73).

In some or any embodiments, the anti-sclerostin antibody binds to a sclerostin polypeptide comprising amino acids 89-103 and 137-151 of SEQ ID NO: 1.

In some or any embodiments, the anti-sclerostin antibody binds to a sclerostin polypeptide having the amino acid sequences of SEQ ID NO:2, SEQ ID NO:3, SEQ ID NO:4 and SEQ ID NO:5, wherein SEQ ID NO:2 and 4 are joined by a disulfide bond at amino acid positions 57 and 111 with reference to SEQ ID NO:1, and SEQ ID NO:3 and 5 are joined by at least one of (a) a disulfide bond at amino acid positions 82 and 142 with reference to SEQ ID NO:1, and (b) a disulfide bond at amino acid positions 86 and 144 with reference to SEQ ID NO:1; the polypeptide may retain the tertiary structure of the corresponding polypeptide region of human sclerostin of SEQ ID NO:1. Alternatively or in addition, the anti-sclerostin antibody binds a polypeptide having the amino acid sequences of SEQ ID NO: 70, SEQ ID NO: 71, SEQ ID NO: 72 and SEQ ID NO: 73, wherein SEQ ID NO: 72 and 73 are joined by a disulfide bond at amino acid positions 57 and 111 with reference to SEQ ID NO: 1, and SEQ ID NO: 70 and 71 are joined by at least one of (a) a disulfide bond at amino acid positions 82 and 142 with reference to SEQ ID NO: 1, and (b) a disulfide bond at amino acid positions 86 and 144 with reference to SEQ ID NO: 1.

Optionally, the anti-sclerostin antibody binds a peptide consisting essentially of the amino acid sequences of SEQ ID NO: 2, SEQ ID NO: 3, SEQ ID NO: 4 and SEQ ID NO: 5, wherein SEQ ID NO: 2 and 4 are joined by a disulfide bond at amino acid positions 57 and 111 with reference to SEQ ID NO: 1, and SEQ ID NO: 3 and 5 are joined by at least one of (a) a disulfide bond at amino acid positions 82 and 142 with reference to SEQ ID NO: 1, and (b) a disulfide bond at amino acid positions 86 and 144 with reference to SEQ ID NO: 1.

Optionally, the anti-sclerostin antibody binds to a polypeptide consisting essentially of a multiply truncated human sclerostin protein of SEQ ID NO: 1, wherein (a) amino acids 1-50, 65-72, 91-100, 118-137, and 150-190 of SEQ ID NO: 1 are absent from said polypeptide or (b) amino acids 1-56, 65-72, 87-110, 118-137, and 145-190 of SEQ ID NO: 1 are absent from said polypeptide.

In some or any embodiments, the anti-sclerostin antibody binds to a polypeptide having the amino acid sequences of SEQ ID NO: 70, SEQ ID NO: 71, SEQ ID NO: 72 and SEQ ID NO: 73, wherein SEQ ID NO: 72 and 73 are joined by a disulfide bond at amino acid positions 57 and 111 with reference to SEQ ID NO: 1, and SEQ ID NO: 70 and 71 are joined by at least one of (a) a disulfide bond at amino acid positions 82 and 142 with reference to SEQ ID NO: 1, and (b) a disulfide bond at amino acid positions 86 and 144 with reference to SEQ ID NO: 1.

In some or any embodiments, the sclerostin polypeptide retains the tertiary structure of the corresponding polypeptide region of human sclerostin of SEQ ID NO: 1.

In some or any embodiments, the anti-sclerostin antibody binds to (i) a portion of human sclerostin comprising amino acids 51-64, 73-90, 101-117, and 138-149 of SEQ ID NO: 1, wherein said portion has at least one, at least two or all three of: (a) a disulfide bond between amino acids 57 and 111; (b) a disulfide bond between amino acids 82 and 142; and (c) a disulfide bond between amino acids 86 and 144; or (ii) a portion of human sclerostin comprising amino acids 57-64, 73-86, 111-117, and 138-144 of SEQ ID NO: 1, wherein said portion has at least one, at least two, or all three of: (a) a disulfide bond between amino acids 57 and 111; (b) a disulfide bond between amino acids 82 and 142; and (c) a disulfide bond between amino acids 86 and 144.

In some or any embodiments, the anti-sclerostin antibody also binds to an epitope of SEQ ID NO: 6.

Anti-sclerostin antibodies preferably modulate sclerostin function in the cell-based assay described in U.S. Patent Publication No. 2007/0110747 and/or the in vivo assay described in U.S. Patent Publication No. 2007/0110747 and/or bind to one or more of the epitopes described in U.S. Patent Publication No. 2007/0110747 and/or cross-block the binding of one of the antibodies described in U.S. Patent Publication No. 2007/0110747 and/or are cross-blocked from binding sclerostin by one of the antibodies described in U.S. Patent Publication No. 2007/0110747 (incorporated by reference in its entirety and for its description of assays for characterizing an anti-sclerostin antibody).

In various aspects, the anti-sclerostin antibody is also capable of neutralizing human sclerostin in a MC3T3 cell-based mineralization assay when there is less than a 6-fold excess of moles of sclerostin binding sites per well as compared to the number of moles of sclerostin per well. Mineralization by osteoblast-lineage cells in culture, either primary cells or cell lines, is used as an in vitro model of bone formation. An exemplary cell-based mineralization assay is described in U.S. Patent Publication No. 20070110747 at, e.g., Example 8 (hereby incorporated by reference). MC3T3-E1 cells (Sudo et al., J. Cell Biol., 96:191-198 (1983)) and subclones of the original cell line can form mineral in culture upon growth in the presence of differentiating agents. Such subclones include MC3T3-E1-BF (Smith et al., J. Biol. Chem., 275:19992-20001 (2000)). For both the MC3T3-E1-BF subclone as well as the original MC3T3-E1 cells, sclerostin can inhibit one or more of the sequence of events leading up to and including mineral deposition (i.e., sclerostin inhibits mineralization). Anti-sclerostin antibodies that are able to neutralize sclerostin's inhibitory activity allow for mineralization of the culture in the presence of sclerostin such that there is a statistically significant increase in, e.g., deposition of calcium phosphate (measured as calcium) as compared to the amount of calcium measured in the sclerostin-only (i.e., no antibody) treatment group.

When running the assay with the goal of determining whether a particular anti-sclerostin antibody (or other sclerostin inhibitor) can neutralize sclerostin, the amount of sclerostin used in the assay desirably is the minimum amount of sclerostin that causes at least a 70%, statistically significant, reduction in deposition of calcium phosphate (measured as calcium) in the sclerostin-only group, as compared to the amount of calcium measured in the no sclerostin group. An anti-sclerostin neutralizing antibody is defined as one that causes a statistically significant increase in deposition of calcium phosphate (measured as calcium) as compared to the amount of calcium measured in the sclerostin-only (i.e., no antibody) treatment group. To determine whether an anti-sclerostin antibody is neutralizing or not, the amount of anti-sclerostin antibody used in the assay is such that there is an excess of moles of sclerostin binding sites per well as compared to the number of moles of sclerostin per well. Depending on the potency of the antibody, the fold excess that may be required can be 24, 18, 12, 6, 3, or 1.5, and one of skill is familiar with the routine practice of testing more than one concentration of binding agent (antibody). For example, a very potent anti-sclerostin neutralizing antibody will neutralize sclerostin when there is less than a 6-fold excess of moles of sclerostin binding sites per well as compared to the number of moles of sclerostin per well. A less potent anti-sclerostin neutralizing antibody will neutralize sclerostin only at a 12, 18 or 24 fold excess.

The anti-sclerostin antibody optionally has an IC50 of 100 nM or less, or 75 nM or less, or 50 nM or less, or 25 nM or less for neutralizing human sclerostin in a cell-based assay, such as a bone specific alkaline phosphatase assay, e.g., the bone specific alkaline phosphatase assay described in International Patent Publication No. WO 2008/115732 and U.S. Pat. No. 7,744,874 (incorporated herein by reference in its entirety for its description of cell-based assays and anti-sclerostin antibodies). The bone specific alkaline phosphatase assay is predicated on the ability of sclerostin to decrease BMP-4 and Wnt3a-stimulated alkaline phosphatase levels in the multipotential murine cell line, C2C12. According to WO 2008/115732, a neutralizing anti-sclerostin antibody mediates a dose-dependent increase of alkaline phosphatase activity in this assay.

Alternatively or in addition, the anti-sclerostin antibody has an IC50 of 100 nM or less (e.g., 75 nM or less, or 50 nM or less) for neutralizing human sclerostin in a cell-based Wnt signaling assay in HEK293 cell lines, such as the Wnt assay involving Wntl-mediated induction of STF reporter gene described in e.g., International Patent Publication No. WO 2009/047356 (incorporated by reference for its discussion of anti-sclerostin antibodies and cell-based assays). Alternatively or in addition, the anti-sclerostin antibody has an IC50 of 500 nM or less (e.g., 250 nM or less, 150 nM or less, 100 nM or less, or 50 nM or less) for neutralizing human sclerostin in a BMP2-induced mineralization assay in MC3T3 cells, such as the mineralization assay described in e.g., International Patent Publication No. WO 2009/047356.

Examples of anti-sclerostin antibodies suitable for use in the context of the invention are described in U.S. Patent Publication Nos. 2007/0110747 and 2007/0072797, which are hereby incorporated by reference. In some embodiments, the anti-sclerostin antibody cross-blocks the binding of at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24 (all of which are described in U.S. Patent Publication No. 20070110747) to sclerostin. Alternatively or in addition, the anti-sclerostin antibody is cross-blocked from binding to sclerostin by at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24 (all of which are described in U.S. Patent Publication No. 20070110747). The terms “cross-block,” “cross-blocked,” and “cross-blocking” are used interchangeably herein to mean the ability of an antibody to interfere with the binding of other antibodies to sclerostin. The extent to which an antibody is able to interfere with the binding of another to sclerostin, and therefore whether it can be said to cross-block, can be determined using competition binding assays. In some aspects of the invention, a cross-blocking antibody or fragment thereof reduces sclerostin binding of a reference antibody between about 40% and about 100%, such as about 60% and about 100%, specifically between 70% and 100%, and more specifically between 80% and 100%. A particularly suitable quantitative assay for detecting cross-blocking uses a Biacore machine which measures the extent of interactions using surface plasmon resonance technology. Another suitable quantitative cross-blocking assay uses an ELISA-based approach to measure competition between antibodies in terms of their binding to sclerostin.

In some embodiments, the anti-sclerostin antibody cross-blocks the binding of an immunoglobulin comprising full length heavy and light chains to sclerostin of SEQ ID NO: 1 and/or is cross-blocked from binding to sclerostin of SEQ ID NO: 1 by an immunoglobulin comprising full length heavy and light chains, wherein the immunoglobulin comprising full length heavy and light chains comprise CDR sequences disclosed herein, such as one of the following three sets of CDR sequences: a) CDR-L1 of SEQ ID NO: 284, CDR-L2 of SEQ ID NO: 285, CDR-L3 of SEQ ID NO: 286, CDR-H1 of SEQ ID NO: 296, CDR-H2 of SEQ ID NO: 297, and CDR-H3 of SEQ ID NO: 298; b) CDR-L1 of SEQ ID NO: 48, CDR-L2 of SEQ ID NO: 49, CDR-L3 of SEQ ID NO: 50, CDR-H1 of SEQ ID NO: 45, CDR-H2 of SEQ ID NO: 46, and CDR-H3 of SEQ ID NO: 47; or c) CDR-L1 of SEQ ID NO: 42, CDR-L2 of SEQ ID NO: 43, CDR-L3 of SEQ ID NO: 44, CDR-H1 of SEQ ID NO: 39, CDR-H2 of SEQ ID NO: 40, and CDR-H3 of SEQ ID NO: 41. Alternatively, or in addition, the anti-sclerostin antibody cross-blocks the binding of immunoglobulin comprising full length heavy and light chains to sclerostin of SEQ ID NO: 1 and/or is cross-blocked from binding to sclerostin of SEQ ID NO: 1 by an immunoglobulin comprising full length heavy and light chains, wherein the immunoglobulin comprising full length heavy and light chains comprise the following CDRs: CDR-H1 of SEQ ID NO: 245, CDR-H2 of SEQ ID NO: 246, CDR-H3 of SEQ ID NO: 247, CDR-L1 of SEQ ID NO: 78, CDR-L2 of SEQ ID NO: 79 and CDR-L3 of SEQ ID NO: 80.

Alternatively, or in addition, the anti-sclerostin antibody cross-blocks the binding of immunoglobulin comprising full length heavy and light chains to sclerostin of SEQ ID NO: 1 and/or is cross-blocked from binding to sclerostin of SEQ ID NO: 1 by an immunoglobulin comprising full length heavy and light chains, wherein the immunoglobulin comprising full length heavy and light chains comprise the following CDRs: CDR-H1 of SEQ ID NO: 269, CDR-H2 of SEQ ID NO: 270, CDR-H3 of SEQ ID NO: 271, CDR-L1 of SEQ ID NO: 239, CDR-L2 of SEQ ID NO: 240 and CDR-L3 of SEQ ID NO: 241.

Examples of suitable anti-sclerostin antibodies and fragments thereof include antibodies and antibody fragments having one or more of CDR-H1, CDR-H2, CDR-H3, CDR-L1, CDR-L2 and CDR-L3 specifically disclosed herein and disclosed in U.S. Patent Publication No. 2007/0110747. At least one of the regions of CDR-H1, CDR-H2, CDR-H3, CDR-L1, CDR-L2, and CDR-L3 may have at least one amino acid substitution, provided that the antibody retains the binding specificity of the non-substituted CDR. Exemplary the anti-sclerostin antibodies include, but are not limited to, Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24 of U.S. Patent Publication No. 2007/0110747. Other exemplary anti-sclerostin antibodies include, but are not limited to, 27H6, 19D11 and 20C3.

In addition, the anti-sclerostin antibody can comprise at least one CDR sequence having at least 75% identity (e.g., 100% identity) to a CDR selected from SEQ ID NOs: 39, 40, 41, 42, 43, 44, 45, 46, 47, 48, 49, 50, 51, 52, 53, 54, 55, 56, 57, 58, 59, 60, 61, 62, 78, 79, 80, 81, 99, 100, 101, 102, 103, 104, 105, 106, 107, 108, 109, 110, 111, 112, 113, 114, 115, 116, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 351, 352, 353, 358, 359, and 360. In addition, the anti-sclerostin antibody can comprise at least one CDR sequence having at least 75% identity (e.g., 100% identity) to a CDR selected from SEQ ID NOs: 417-422, 425-430 and 433-438 provided in the Sequence Listing. Preferably, the anti-sclerostin antibody comprises at least one CDR sequence having at least 75% identity to a CDR selected from SEQ ID NOs: 245, 246, 247, 78, 79, 80, 269, 270, 271, 239, 240, and 241. The anti-sclerostin antibody can comprise: a) CDR sequences of SEQ ID NOs:54, 55, and 56 and CDR sequences of SEQ ID NOs:51, 52, and 53; b) CDR sequences of SEQ ID NOs:60, 61, and 62 and CDR sequences of SEQ ID NOs:57, 58, and 59; c) CDR sequences of SEQ ID NOs:48, 49, and 50 and CDR sequences of SEQ ID NOs:45, 46, and 47; d) CDR sequences of SEQ ID NOs:42, 43, and 44 and CDR sequences of SEQ ID NOs:39, 40, and 41; e) CDR sequences of SEQ ID NOs:275, 276, and 277 and CDR sequences of SEQ ID NOs:287, 288, and 289; f) CDR sequences of SEQ ID NOs:278, 279, and 280 and CDR sequences of SEQ ID NOs:290, 291, and 292; g) CDR sequences of SEQ ID NOs:78, 79, and 80 and CDR sequences of SEQ ID NOs: 245, 246, and 247; h) CDR sequences of SEQ ID NOs:81, 99, and 100 and CDR sequences of SEQ ID NOs:248, 249, and 250; i) CDR sequences of SEQ ID NOs:101, 102, and 103 and CDR sequences of SEQ ID NOs:251, 252, and 253; j) CDR sequences of SEQ ID NOs:104, 105, and 106 and CDR sequences of SEQ ID NOs:254, 255, and 256; k) CDR sequences of SEQ ID NOs:107, 108, and 109 and CDR sequences of SEQ ID NOs:257, 258, and 259; 1) CDR sequences of SEQ ID NOs:110, 111, and 112 and CDR sequences of SEQ ID NOs:260, 261, and 262; m) CDR sequences of SEQ ID NOs:281, 282, and 283 and CDR sequences of SEQ ID NOs:293, 294, and 295; n) CDR sequences of SEQ ID NOs:113, 114, and 115 and CDR sequences of SEQ ID NOs:263, 264, and 265; o) CDR sequences of SEQ ID NOs:284, 285, and 286 and CDR sequences of SEQ ID NOs:296, 297, and 298; p) CDR sequences of SEQ ID NOs:116, 237, and 238 and CDR sequences of SEQ ID NOs:266, 267, and 268; q) CDR sequences of SEQ ID NOs:239, 240, and 241 and CDR sequences of SEQ ID NOs:269, 270, and 271) CDR sequences of SEQ ID NOs:242, 243, and 244 and CDR sequences of SEQ ID NOs:272, 273, and 274; or s) CDR sequences of SEQ ID NOs:351, 352, and 353 and CDR sequences of SEQ ID NOs:358, 359, and 360.

The anti-sclerostin antibody can comprise at least one CDR sequence having at least 75% identity (e.g., 100% identical) to a CDR selected from CDR-H1, CDR-H2, CDR-H3, CDR-L1, CDR-L2, and CDR-L3 wherein CDR-H1 has the sequence given in SEQ ID NO: 245, CDR-H2 has the sequence given in SEQ ID NO: 246, CDR-H3 has the sequence given in SEQ ID NO: 247, CDR-L1 has the sequence given in SEQ ID NO: 78, CDR-L2 has the sequence given in SEQ ID NO: 79 and CDR-L3 has the sequence given in SEQ ID NO: 80. The anti-sclerostin antibody, in various aspects, comprises two of the CDRs or six of the CDRs. Optionally, the anti-sclerostin antibody comprises all or part of a heavy chain (e.g., two heavy chains) comprising SEQ ID NO: 378 and all or part of a light chain (e.g., two light chains) comprising SEQ ID NO 376.

The anti-sclerostin antibody can comprise at least one CDR sequence having at least 75% identity (e.g., 100% identical) to a CDR selected from CDR-H1, CDR-H2, CDR-H3, CDR-L1, CDR-L2, and CDR-L3 wherein CDR-H1 has the sequence given in SEQ ID NO: 269, CDR-H2 has the sequence given in SEQ ID NO: 270, CDR-H3 has the sequence given in SEQ ID NO: 271, CDR-L1 has the sequence given in SEQ ID NO: 239, CDR-L2 has the sequence given in SEQ ID NO: 240 and CDR-L3 has the sequence given in SEQ ID NO 241. The anti-sclerostin antibody, in various aspects, comprises at least two of the CDRs or six of the CDRs. Optionally, the anti-sclerostin antibody comprises all or part of a heavy chain (e.g., two heavy chains) comprising SEQ ID NO: 366 and all or part of a light chain (e.g., two light chains) comprising SEQ ID NO 364.

Alternatively, the anti-sclerostin antibody can have a heavy chain comprising CDR's H1, H2, and H3 and comprising a polypeptide having the sequence provided in SEQ ID NO: 137, 145, or 392 or a variant thereof in which the CDRs are at least 75% identical (e.g., 100% identical) to SEQ ID NO: 245, 246, and 247, respectively, and a light chain comprising CDR's L1, L2 and L3 and comprising a polypeptide having the sequence provided in SEQ ID NO: 133 or 141 or a variant thereof in which the CDRs are at least 75% identical (e.g., 100% identical) to SEQ ID NO: 78, 79, and 80, respectively.

The anti-sclerostin antibody may have a heavy chain comprising CDR's H1, H2, and H3 and comprising a polypeptide having the sequence provided in SEQ ID NO: 335, 331, 345, or 396 or a variant of any of the foregoing in which the CDRs are at least 75% (e.g., 100% identical) identical to SEQ ID NO: 269, 270, and 271, respectively, and a light chain comprising CDR's L1, L2, and L3 and comprising a polypeptide having the sequence provided in SEQ ID NO: 334 or 341 or a variant of any of the foregoing in which the CDRs are at least 75% identical (e.g., 100% identical) to SEQ ID NO: 239, 240, and 241, respectively. All combinations of the heavy and light chain sequences are contemplated (e.g., heavy chains comprising SEQ ID NO: 335 and light chains comprising SEQ ID NO: 334; heavy chains comprising SEQ ID NO: 331 and light chains comprising SEQ ID NO: 334 or 341; and heavy chains comprising SEQ ID NO: 345 or 396 and light chains comprising SEQ ID NO: 341).

Alternatively, the anti-sclerostin antibody has a heavy chain comprising a polypeptide having the sequence provided in SEQ ID NO:137, and a light chain comprising a polypeptide having the sequence provided in SEQ ID NO:133; a heavy chain comprising a polypeptide having the sequence provided in SEQ ID NO:145 or 392, and a light chain comprising a polypeptide having the sequence provided in SEQ ID NO: 141; a heavy chain comprising a polypeptide having the sequence provided in SEQ ID NO:335, and a light chain comprising a polypeptide having the sequence provided in SEQ ID NO:334; a heavy chain comprising a polypeptide having the sequence provided in SEQ ID NO:331, and a light chain comprising a polypeptide having the sequence provided in SEQ ID NO:341; or a heavy chain comprising a polypeptide having the sequence provided in SEQ ID NO:345 or 396, and a light chain comprising a polypeptide having the sequence provided in SEQ ID NO:341. Alternatively, the anti-sclerostin antibody cross-blocks (or is cross-blocked by) any of the aforementioned antibodies to sclerostin.

In some embodiments, the anti-sclerostin antibody comprises a heavy chain that comprises an amino acid sequence selected from the group consisting of SEQ ID NO: 1038, SEQ ID NO: 1046, SEQ ID NO: 1040 and SEQ ID NO: 1048; optionally further comprising a light chain amino acid sequence selected from the group consisting of SEQ ID NO: 1039, SEQ ID NO: 1047, SEQ ID NO: 1041 and SEQ ID NO: 1049.

Examples of anti-sclerostin antibodies also include, but are not limited to, the anti-sclerostin antibodies disclosed in International Patent Publication Nos. WO 2008/092894, WO 2008/115732, WO 2009/056634, WO 2009/047356, WO 2010/100200, WO 2010/100179, WO 2010/115932, and WO 2010/130830 (each of which is incorporated by reference herein in its entirety), such as an anti-sclerostin antibody comprising CDRs of SEQ ID NOs: 20-25 of International Patent Publication No. WO 2008/115732 (SEQ ID NOs: 416-421 herein), an anti-sclerostin antibody comprising CDRs of SEQ ID NOs: 26-31 of International Patent Publication No. WO 2008/115732 (SEQ ID NOs: 422-427 herein), an anti-sclerostin antibody comprising CDRs of SEQ ID NOs: 32-37 of International Patent Publication No. WO 2008/115732 (SEQ ID NOs: 428-433 herein), an anti-sclerostin antibody comprising CDRs of SEQ ID NOs: 4, 15, 26, 37, 48, and 59 of International Patent Publication No. WO 2009/047356 (SEQ ID NOs: 443, 454, 465, 476, 487 and 498, respectively, herein), or an anti-sclerostin antibody comprising the amino acid sequence of at least one of SEQ ID NOs: 135-143, 153-161, or 171-179 of International Patent Publication No. WO 2010/130830 (SEQ ID NOs: 745-753, 763-771, 781-789, respectively, herein).

Timing of Administration and Dosage

In some embodiments, one or more administrations of an anti-sclerostin antibody described herein are carried out over a therapeutic period of, for example, about 1 week to about 18 months (e.g., about 1 month to about 12 months, about 1 month to about 9 months or about 1 month to about 6 months or about 1 month to about 3 months). In some embodiments, a subject is administered one or more doses of an anti-sclerostin antibody described herein over a therapeutic period of, for example about 1 month to about 12 months (52 weeks) (e.g., about 2 months, about 3 months, about 4 months, about 5 months, about 6 months, about 7 months, about 8 months, about 9 months, about 10 months, or about 11 months). In some embodiments, a subject is administered one or more doses of the anti-sclerostin antibody to maintain bone mineral density and/or enhance connective tissue-to-bone attachment. The term “maintain bone mineral density” as used herein means that the increased bone mineral density resulting from the initial dose of the anti-sclerostin antibody does not fall more than about 1% to about 5% over the course of about 6 months, about 9 months about 1 year, about 18 months, about 2 years, or over the course of the patient's life). It will be appreciated that a patient can require alternate treatment phases for increasing bone density and maintaining bone density. Enhanced connective tissue-to-bone attachment in a subject receiving the anti-sclerostin antibody can be assessed in a variety of ways, include, but not limited to a perceived reduction in pain, ability of the subject to utilize affected muscle earlier in the healing process, improved radiographic or MRI parameters, and/or increased muscle strength.

In addition, it may be advantageous to administer multiple doses of the anti-sclerostin antibody or space out the administration of doses, depending on the therapeutic regimen selected for a particular subject. In some embodiments, the anti-sclerostin antibody or fragment thereof is administered periodically over a time period of one year (12 months, 52 weeks) or less (e.g., 9 months or less, 6 months or less, or 3 months or less). In this regard, the anti-sclerostin antibody or fragment thereof is administered to the human once every about 3 days, or about 7 days, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 6 months, or 12 months.

In some embodiments, the therapeutic period begins soon after a defect in connective tissue attachment to bone is detected (or soon after surgical reattachment of the connective tissue to bone), e.g., within 30 minutes, within 1 hour, within 2 hours, within 6 hours, within 12 hours or within 24 hours of the defect. In other embodiments, the inhibitor is administered within 1 day of the defect, within 3 days of the defect, within 5 days of the defect, within 7 days of the defect, or within two weeks of the defect, wherein the anti-sclerostin antibody or fragment thereof is administered for a period of time that is at least 4 weeks post defect (e.g., 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks 19 weeks, 20 weeks, 21 weeks, 22 weeks, 23 weeks, 24 weeks, 25 weeks, 26 weeks, 27 weeks 28 weeks, 29 weeks, 30 weeks, 31 weeks or longer (e.g., 8 months, 9 months, 10 months, 11 months, 1 year, 18 months or longer)). In other embodiments, the inhibitor is administered within 1 day of the surgical reattachment, within 3 days of the surgical reattachment, within 5 days of the surgical reattachment, within 7 days of the surgical reattachment, or within two weeks of the surgical reattachment, wherein the anti-sclerostin antibody or fragment thereof is administered for a period of time that is at least 4 weeks post surgical reattachment (e.g., 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks 19 weeks, 20 weeks, 21 weeks, 22 weeks, 23 weeks, 24 weeks, 25 weeks, 26 weeks, 27 weeks 28 weeks, 29 weeks, 30 weeks, 31 weeks or longer (e.g., 8 months, 9 months, 10 months, 11 months, 1 year, 18 months or longer)).

In some embodiments, one or more doses of the anti-sclerostin antibody or fragment thereof are administered in an amount and for a time effective to enhance connective tissue-to-bone healing and/or improve the outcome of a connective tissue reattachment procedure. In various embodiments, one or more doses comprising from about 50 milligrams to about 1,000 milligrams of the anti-sclerostin antibody are administered per week to a subject (e.g., a human subject). For example, a dose of anti-sclerostin antibody can comprise at least about 5 mg, 15 mg, 25 mg, 50 mg, about 60 mg, about 70 mg, about 80 mg, about 90 mg, about 100 mg, about 120 mg, about 150 mg, about 200 mg, about 240 mg, about 250 mg, about 280 mg, about 300 mg, about 350 mg, about 400 mg, about 420 mg, about 450 mg, about 500 mg, about 550 mg, about 600 mg, about 650 mg, about 700 mg, about 750 mg, about 800 mg, about 850 mg, about 900 mg, about 950 mg or up to about 1,000 mg of anti-sclerostin antibody. Ranges between any and all of these endpoints are also contemplated, e.g. about 50 mg to about 80 mg, about 70 mg to about 140 mg, about 70 mg to about 270 mg, about 75 mg to about 100 mg, about 100 mg to about 150 mg, about 140 mg to about 210 mg, or about 150 mg to about 200 mg, or about 180 mg to about 270 mg, or about 280 to about 410 mg. The dose is administered at any interval, such as multiple times a week (e.g., twice or three times per week), once a week, once every two weeks, once every three weeks, or once every four weeks. In some or any embodiments, a dose of anti-sclerostin antibody ranging from about 120 mg to about 210 mg is administered twice a week. In some or any embodiments, a dose of about 140 mg of the anti-sclerostin antibody is administered twice a week.

In some embodiments, the one or more doses of anti-sclerostin antibody can comprise between about 0.1 to about 50 milligrams (e.g., between about 5 and about 50 milligrams), or about 1 to about 100 milligrams, of anti-sclerostin antibody per kilogram of body weight (mg/kg). For example, the dose of anti-sclerostin antibody may comprise at least about 0.1 mg/kg, 0.5 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, or about 49 mg/kg, or about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or up to about 100 mg/kg. Ranges between any and all of these endpoints are also contemplated, e.g., about 1 mg/kg to about 3 mg/kg, about 1 mg/kg to about 5 mg/kg, about 1 mg/kg to about 8 mg/kb, about 3 mg/kg to about 8 mg·kg, about 1 mg/kg to about 10 mg/kg, about 1 mg/kg to about 20 mg/kg, about 1 mg/kg to about 40 mg/kg, about 5 mg/kg to about 30 mg/kg, or about 5 mg/kg to about 20 mg/kg.

Combination Therapy

Treatment of a pathology by combining two or more agents that target the same pathogen or biochemical pathway or biological process sometimes results in greater efficacy and diminished side effects relative to the use of a therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone). As used herein, the term “combination therapy” means that two or more agents are delivered in a simultaneous manner, e.g., concurrently, or wherein one of the agents is administered first, followed by the second agent, e.g., sequentially.

In some embodiments, the anti-sclerostin antibody is administered along with a standard of care therapeutic for the treatment of defects in connective tissue attachment to bone (i.e., the anti-sclerostin antibody and standard of care therapeutic are part of the same treatment plan). As used herein, the term “standard of care” refers to a treatment that is generally accepted by clinicians for a certain type of patient diagnosed with a type of illness. In some embodiments, the anti-sclerostin antibody is administered along with a second bone-enhancing agent useful for the treatment of decreased bone mineral density or bone defect. In some embodiments, the bone-enhancing agent is selected from the group consisting of an anti-resorptive agent, a bone-forming agent (i.e., anabolic), an estrogen receptor modulator (including, but not limited to, raloxifene, bazedoxifene and lasofoxifene) and a drug that has an inhibitory effect on osteoclasts. In some embodiments, the second bone-enhancing agent is selected from the group consisting of a bisphosphonate (including, but not limited to, alendronate sodium (FOSAMAX®), risedronate, ibandronate sodium (BONIVA®) and zoledronic acid (RECLAST®)); an estrogen or estrogen analogue; an anti-RANK ligand (RANKL) inhibitor, such as an anti-RANKL antibody (e.g., PROLIA®); vitamin D, or a vitamin D derivative or mimic thereof; a calcium source, a cathepsin-K (cat-K) inhibitor (e.g. odanacatib), Tibolone, calcitonin or a calcitriol; and hormone replacement therapy. In some embodiments, the second bone-enhancing agent includes, but is not limited to, parathyroid hormone (PTH) or a peptide fragment thereof, PTH-related protein (PTHrp), bone morphogenetic protein, osteogenin, NaF, a PGE2 agonist, a statin, strontium ranelate, a sclerostin inhibitor (e.g., an anti-sclerostin antibody described in, for example, U.S. Pat. No. 7,592,429 or 7,872,106), and an anti-DKK1 antibody or inhibitor. In some embodiments, the second bone-enhancing agent is Forteo® (Teriparatide), Preotact®, or Protelos®. In some embodiments, the second bone-enhancing agent comprises a bone morphogenetic protein (e.g., BMP-1, BMP-2, BMP-3, BMP-4, BMP-5, BMP-6, BMP-7, BMP-8, BMP-9, BMP-10, BMP-11, BMP-12, BMP-13, BMP-14 and/or BMP-15).

Combining anti-sclerostin antibody treatment with a standard of care therapeutic regimen for a connective tissue to bone injury is also contemplated. Exemplary standard of care therapeutics or therapeutic regimens for connective tissue to bone injury include, but are not limited to, bone marrow aspirate, platelet rich plasma, gene therapy (e.g., bFGF, BMP 12-14, PDGF, IGF, TGFβ, CTGF and VEGF), growth factory therapy (e.g., BMP2/Smad8, BMP12/TGFβ1), stem cell therapy (e.g., bone marrow mesenchymal stromal cells, adipose mesenchymal stromal cells, embryonic stem cell derived mesenchymal stromal cells, tendon-derived cells) and the use of natural biomaterials (e.g., collagen-based scaffolds, aligned collagen threads, decellularized tendon grafts and dermis grafts).

In some embodiments, the combination therapy employing a anti-sclerostin antibody described herein may precede or follow administration of additional therapeutic(s) (e.g., second bone-enhancing agent) by intervals ranging from minutes to weeks to months. For example, separate modalities are administered within about 24 hours of each other, e.g., within about 6-12 hours of each other, or within about 1-2 hours of each other, or within about 10-30 minutes of each other. In some situations, it may be desirable to extend the time period for treatment significantly, where several days (2, 3, 4, 5, 6 or 7 days) to several weeks (1, 2, 3, 4, 5, 6, 7 or 8 weeks) lapse between the respective administrations of different modalities. Repeated treatments with one or both agents/therapies of the combination therapy is specifically contemplated.

Maintenance Therapeutic Regimen

Also contemplated is the use of a second bone-enhancing agent and/or anti-sclerostin antibody described herein in a maintenance regimen to, e.g., maintain improved connective tissue-to-bone attachment and/or prevent unloading-induced bone loss. In this regard, a method or use described herein optionally comprises administering one or more amounts of a second bone-enhancing agent effective to maintain improved connective tissue-to-bone attachment for a maintenance period of about 1 week to about 5 years after the treatment period with the anti-sclerostin antibody has ended. For example, in some embodiments, a method or use described herein comprises the administration of a second bone-enhancing agent to the subject for a maintenance period of about at least about 1 week, 2 weeks, 3 weeks, 4 weeks, 5 weeks, 6 weeks, 7 weeks, 8 weeks, 9 weeks, 10 weeks, 11 weeks, 12 weeks, 3 months, 13 weeks, 14 weeks, 15 weeks, 16 weeks, 4 months, 17 weeks, 18 weeks, 19 weeks, 20 weeks, 5 months, 21 weeks, 22 weeks, 23 weeks, 24 weeks, 6 months, 25 weeks, 26 weeks, 27 weeks 28 weeks, 7 months, 29 weeks, 30 weeks, 31 weeks or longer (e.g., 8 months, 9 months, 10 months, 11 months, 1 year, 15 months, 18 months, 2 years, 3 years, 4 years, 5 years or longer (e.g., over the lifetime of the subject). In some embodiments, the maintenance period is about 6-12 weeks. In some embodiments, the maintenance period is about 4-12 weeks, or about 1-3 months. In some embodiments, the maintenance period is about 12-20 weeks, or about 3-5 months. In some embodiments, the maintenance period is about 20-32 weeks, or about 5-8 months. In some embodiments, the maintenance period is about 24-36 weeks, or about 6-9 months. In some embodiments, the maintenance period is about 1 year, about 2 years, about 3 years, about 4 years, about 5 years or longer. “Maintaining” improved connective tissue-to-bone attachment includes maintaining similar levels of radiographic or MRI parameters and/or muscle strength measurements experienced in the subject that received the anti-sclerostin antibody treatment.

Similarly, a method or use described herein optionally comprises subsequently administering one or more amounts of an anti-sclerostin antibody effective to maintain improved connective tissue-to-bone attachment for a maintenance period of at least about least about 1 week, 2 weeks, 3 weeks, 4 weeks, 5 weeks, 6 weeks, 7 weeks, 8 weeks, 9 weeks, 10 weeks, 11 weeks, 12 weeks, 3 months, 13 weeks, 14 weeks, 15 weeks, 16 weeks, 4 months, 17 weeks, 18 weeks, 19 weeks, 20 weeks, 5 months, 21 weeks, 22 weeks, 23 weeks, 24 weeks, 6 months, 1 year, 2 years, 3 years, 4 years, 5 years or longer (e.g., over the lifetime of the subject) after the treatment period has ended. In some embodiments, the maintenance period is about 6-12 weeks. In some embodiments, the maintenance period is about 4-12 weeks, or about 1-3 months. In some embodiments, the maintenance period is about 12-20 weeks, or about 3-5 months. In some embodiments, the maintenance period is about 20-32 weeks, or about 5-8 months. In some embodiments, the maintenance period is about 24-36 weeks, or about 6-9 months. In some embodiments, the maintenance period is about 1 year, about 2 year, about 3 years, about 4 years, about 5 years or longer.

Pharmaceutical Compositions

In some embodiments, an anti-sclerostin described is formulated together with a pharmaceutically effective diluents, carrier, solubilizer, emulsifier, preservative, and/or adjuvant. Pharmaceutical compositions include, but are not limited to, liquid, frozen, and lyophilized compositions.

Preferably, formulation materials are nontoxic to recipients at the dosages and concentrations employed. In specific embodiments, pharmaceutical compositions comprising a therapeutically effective amount of anti-sclerostin antibody or fragment thereof are provided.

In some embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In some embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the anti-sclerostin antibody or fragment. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments of the invention, the composition may be prepared for storage by mixing the selected composition having the desired degree of purity with optional formulation agents (REMINGTON'S PHARMACEUTICAL SCIENCES, supra) in the form of a lyophilized cake or an aqueous solution. Further, in some embodiments, the anti-sclerostin antibody or fragment may be formulated as a lyophilizate using appropriate excipients such as sucrose.

In some embodiments, the pharmaceutical formulation comprises 55 mM acetate, 13 mm calcium, 6.0% (w/v) sucrose, 0.006% (w/v) polysorbate 20, at pH 5.2. In some embodiments, the pharmaceutical composition comprises 90 mg/mL anti-sclerostin antibody.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired anti-sclerostin antibody or fragment in a pharmaceutically acceptable vehicle. A particularly suitable vehicle for parenteral injection is sterile distilled water in which the anti-sclerostin antibody or fragment is formulated as a sterile, isotonic solution, properly preserved. In certain embodiments, the preparation involves the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired anti-sclerostin antibody or fragment thereof.

Additional pharmaceutical compositions will be evident to those skilled in the art, including formulations involving antigen binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP133988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP036676; EP088046 and EP143949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Aspects of the invention includes self-buffering anti-sclerostin antibody or fragment formulations, which can be used as pharmaceutical compositions, as described in International Patent Application Publication WO 2006/138181A2 (PCT/US2006/022599), which is incorporated by reference in its entirety herein.

As discussed above, certain embodiments provide anti-sclerostin antibody or fragment compositions, particularly pharmaceutical anti-sclerostin antibody or fragment compositions, that comprise, in addition to the anti-sclerostin antibody or fragment, one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

Kits

A pharmaceutical composition comprising one or more anti-sclerostin antibodies described herein may be placed within containers (e.g., vials or syringes), along with packaging material that provides instructions regarding the use of such pharmaceutical compositions. Generally, such instructions will include a tangible expression describing the anti-sclerostin antibody concentration, as well as within certain embodiments, relative amounts of excipient ingredients or diluents (e.g., water, saline or PBS) that may be necessary to reconstitute the pharmaceutical composition.

### EXAMPLES

Materials and Methods

Animals and Surgery:

Adult male Sprague-Dawley rats were used in this study, as approved by Washington University's Animal Studies Committee. At the time of surgery rats were approximately four months old and weighed at least 350 g. The supraspinatus (SS) tendon was sharply elevated from the humeral head and repaired, as previously described, in both shoulders [25]. A total of 48 rats were surgically operated on and 34 additional rats were used as healthy normal controls (N). Animals were either left untreated (CTL group) or administered sclerostin antibody (Scl-Ab VI, Amgen, Thousand Oaks, Calif.) having CDR-H1 of SEQ ID NO:245, a CDR-H2 of SEQ ID NO:246, a CDR-H3 of SEQ ID NO:247, a CDR-L1 of SEQ ID NO:78, a CDR-L2 of SEQ ID NO:79, and a CDR-L3 of SEQ ID NO:80 via subcutaneous injections (25 mg/kg) at the time of injury-and-repair and every two weeks until sacrifice (Scl-Ab group). All animals were allowed free cage activity and sacrificed after 2, 4, or 8 weeks of healing. Non-injured animals were sacrificed at an average age of 5 months, which corresponds to approximately 4 weeks of healing after injury.

Bone Morphometry:

After sacrifice, the humerus with the supraspinatus tendon and muscle attached was carefully dissected from one shoulder. For bone morphometry (N=17 for non-injured, N=5 for 2 wk healing, N=20 for 4 wk healing, and N=23 for 8 wk healing), the humeral head and tendon enthesis region (˜5 mm) were scanned using micro computed tomography (microCT) at a resolution of 20 μm, 45 kVp, and 177 μA (μCT 40, Scanco Medical, Switzerland), as described previously [15, 26]. The region of interest included trabecular bone within the humeral head near the tendon attachment and proximal to the growth plate. The amount of bone in the region of interest was calculated to determine bone fraction volume (BV/TV). Bone mineral density (BMD), trabecular number (Tb.N), and trabecular thickness (Tb.Th) were also determined.

Biomechanics:

After sacrifice, dissection, and microCT scanning, the supraspinatus muscle was gently removed from the tendon in preparation for testing, as described previously [15]. The repair-site suture was released to remove its mechanical contribution during load-to-failure tensile tests. The growth plates was secured by looping 4/0 surgical steel wire around the humeral head. The humeri were then potted in poly-methyl-methacrylate. Specimens were tested in a 0.9% saline water bath at 37° C. Biomechanical tests were performing using a uniaxial testing frame (Instron 5866, Instron Corporation, Norwood, Mass.) and a thin film tendon grip (Imada, Northbrook, Ill.). Uniaxial load-to-failure tensile tests consisted of 5 cycles of preconditioning to 5% strain at 0.2%/s, followed by a recovery period of 300 s, and extension to failure at 0.2%/s. The strain was measured as grip-to-grip displacement relative to the initial measured gauge length of each tendon. The cross-sectional area (CSA) of the supraspinatus tendon near the attachment site was measured from μCT scans described above. Measured force was divided by CSA to calculate stress. Load-deformation curves were used to determine maximum load and stiffness (slope of the linear portion of the curve). Stress-strain curves were used to determine strength (maximum stress), modulus (slope of the linear portion of the curve) and resilience. The mechanism of failure was determined visually during the test and verified via gross observations after completion of the test.

Histology:

For histology, dissected humerus-supraspinatus constructs were first fixed in 4% paraformaldehyde for 24 hours. Some samples (18 of 24) were decalcified in 14% ethylenediaminetetraacetic acid, dehydrated in graded ethanol, and embedded in paraffin. Coronal sections with thickness of 5 μm were stained with hematoxylin and eosin, toluidine blue, or Goldner's trichrome. The remaining samples (6 of 24) were fixed for 24 hours in 4% paraformaldehyde, embedded in plastic, and 5 μm thick coronal sections were stained with Von Kossa. The sections were semi-quantitatively analyzed by one blinded observer (NH) using a tendon-to-bone maturing score adopted from Ide et al. [26, 27]. Insertion continuity, bone resorption, matrix quality, cell and fiber alignment, and cellularity were part of nine factors evaluated on a scale from 1-4 (Table 1). A lower score is indicative of improved tendon-to-bone healing, with a score of 9 equivalent to a healthy attachment [26, 27].

TABLE 1
The modified tendon-to-bone maturing score involved evaluation of nine
individual outcomes on a scale of 1-4. A healthy (uninjured) enthesis has a combined
score of 9.

| | Tendon to Bone Maturing Score | | | |
|---|---|---|---|---|
| | 1 | 2 | 3 | 4 |
| Cellularity | Minimal | Mild | Moderate | Marked |
| (inflammation) | | | | |
| Presence of | >75% | 51-75% | 26-50% | <25% |
| fibrocytes | | | | |
| Proportion of cells | >75% | 51-75% | 26-50% | <25% |
| oriented parallel | | | | |
| Proportion of fibers | >75% | 51-75% | 26-50% | <25% |
| oriented parallel | | | | |
| Presence of Matrix | Marked | Moderate | Mild | Minimal |
| Insertion Integrity | C(+) R(+) F(+) | C(+) R(+) F(+) | C(+) R(+) F(−) | C(+) R(−) |
| | Tidemark (+) | Tidemark (−) | | |
| Insertion % | >75% | 51-75% | 26-50% | <25% |
| continuity | | | | |
| Bone resorption at | <25% | 26-50% | 51-75% | >75% |
| enthesis | | | | |
| Epiphyseal bone | <25% | 26-50% | 51-75% | >75% |
| modeling | | | | |

C—continuity
R—regularity
F—fibrocartilage.

Gene Expression:

For gene expression studies, dissected humerus-supraspinatus samples were flash frozen in liquid nitrogen. RNA was isolated separately from the supraspinatus tendon and the portion of the humeral head proximal to the growth plate near the tendon attachment (RNAeasy Kit, Qiagen Valencia, Calif.). RNA was quantified using a NanoDrop 2000 (Thermo Scientific, Waltham, Mass.). Quantitative real-time PCR (qRT-PCR) was performed using a Biomark HD System (Fluidigm, San Francisco, Calif.) for tendon and bone RNA on a panel of 25 genes related to bone, tendon, and fibrocartilage (Table 2). TaqMan gene expression assays (Life Technologies, Carlsbad, Calif.) were used for the analysis. Rp113a was used as a housekeeping gene, as expression of Rp113a did not vary more than ±0.5 CT value among groups. Results are presented as relative expression compared to Rp113a expression (2^{-ΔCt}).

TABLE 2
Names of genes and associated category and TaqMan Assay ID
evaluated.

| Gene Name | Category | TaqMan Assay ID |
|---|---|---|
| Osteoprotegrin (OPG), | Bone, inhibits | Rn00563499_m1 |
| Tnfrsf11b | osteoclast activity | |
| Sclerostin (SOST) | Bone, osteoclast | Rn00577971_m1 |
| | activity | |
| Dmp1 | Bone, osteoclast | Rn01450122_m1 |
| | activity | |
| RankL, Tnfsf11 | Bone, osteoblast | Rn00589289_m1 |
| | activity | |
| Osteocalcin (OCN), Bglap | Bone, osteoblast | Rn00566386_g1 |
| | activity | |
| Pthlr | Bone, osteoblast | Rn00571596_m1 |
| | activity | |
| Cathepsin K (Ctsk) | Bone, bone resorption | Rn00580723_m1 |
| Dkk1 | Bone, Wnt signaling | Rn01501537_m1 |
| | antagonist | |
| Lrp5 | Bone, Wnt signaling | Rn01451428_m1 |
| Runx2 | Bone, osteoprogenitor | Rn01512298_m1 |
| Osterix, Sp7 | Bone, osteoprogenitor | Rn02769744_s1 |
| Scleraxis (Scx) | Tendon | Rn01504576_m1 |
| Tenomodulin (Tnmd) | Tendon | Rn00574164_m1 |
| Collagen Type I (Col 1a1) | Tendon | Rn01463848_m1 |
| Collagen Type I (Col 1a2) | Tendon | Rn01526721_m1 |
| Collagen Type III (Col 3a1) | Tendon | Rn01437681_m1 |
| Collagen Type II (Col 2a1) | Fibrocartilage | Rn016370876_m1 |
| Aggrecan (Acan) | Fibrocartilage | Rn00573424_m1 |
| Tgfb1 | Fibrocartilage | Rn00572010_m1 |
| Tgfb3 | Fibrocartilage | Rn00565937_m1 |
| Mmp2 | Fibrocartilage | Rn01538170_m1 |
| Sox9 | Fibrocartilage | Rn01751069_m1 |
| Smoothened (Smo) | Development | Rn00563043_m1 |
| Notch1 | Development | Rn01758633_m1 |
| Rpl 13a | Housekeeping | Rn00821946_g1 |

Statistics:

A two factor analysis of variance (ANOVA; factors Treatment and Healing Time) followed by Tukey's post hoc tests was used to determine effects of treatment and duration of healing. Additional statistical comparisons with the non-injured group were done with 2-tailed student t-test. P<0.05 was considered significant.

Results

Bone Morphometry:

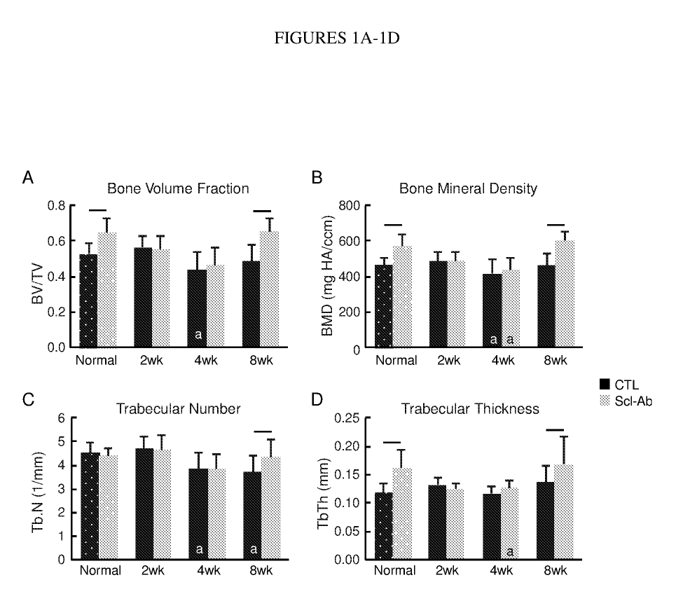

There was significant bone loss in CTL and Scl-Ab groups by 4 weeks of healing, with recovery in the Scl-Ab group by 8 weeks of healing (FIG. 1). Specifically, BV/TV, BMD, and TbN were significantly decreased in CTL and Scl-Ab groups compared to the non-injured group at 4 weeks. However, after 8 weeks of healing, when comparing Scl-Ab treatment to CTL, BV/TV was increased by 34%, BMD was increased by 30%, TbN was increased by 17%, TbTh was increased by 24%, and TbSp was decreased by 21% (FIGS. 1A-1D), reaching levels comparable to those of the non-injured control group. Treatment with Scl-Ab also led to increased BV/TV, BMD, and TbTh in the non-injured groups as well. When evaluating the overall effect of Scl-Ab treatment using an ANOVA, Scl-Ab treated animals had significantly higher BV/TV, BMD, and TbTh compared to CTL animals, by 19%, 18%, and 20%, respectively.

Biomechanics:

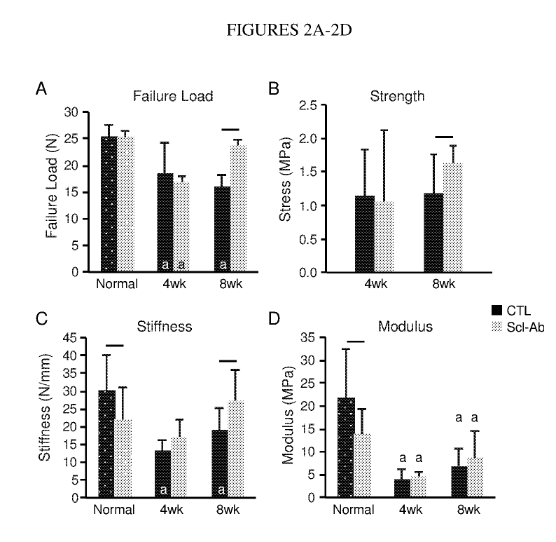

Injury caused a significant increase in CSA (15.9±3.9 mm2 vs. 6.6±1.9 mm²; note that CSA measurements included all soft tissue near the enthesis, including tendon and scar). There was a significant decrease in mechanical properties in CTL and Scl-Ab groups by 4 weeks of healing, with recovery in the Scl-Ab group by 8 weeks of healing (FIG. 2). Specifically, failure load, stiffness, and modulus were significantly decreased in CTL and Scl-Ab groups compared to the non-injured group at 4 weeks. However, after 8 weeks of healing, when comparing Scl-Ab treatment to CTL, failure load was increased by 48%, strength was increased by 38%, and stiffness was increased by 43%. Surprisingly, treatment with Scl-Ab also led to significant decreases in stiffness and modulus in the non-injured groups.

Histology:

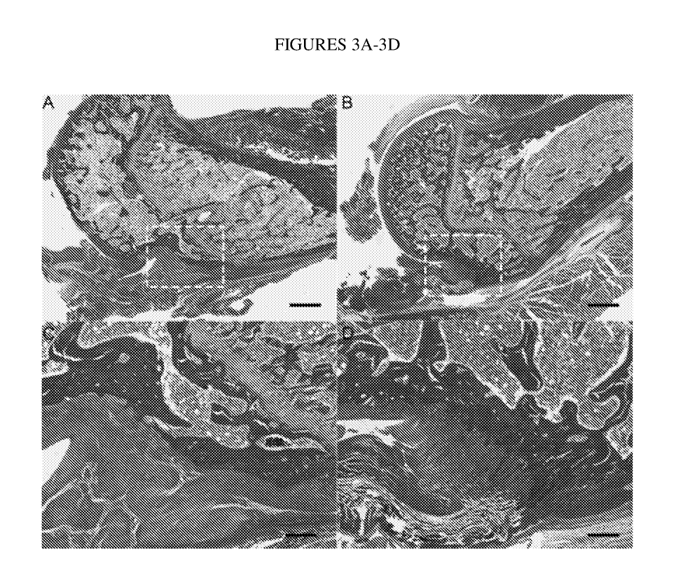

No repair site failures or gaps were noted at the time of dissection and sample preparation. Histological sections demonstrated supraspinatus tendons healing to humeral head bone via fibrovascular scar, with bone loss evident in the humeral heads of the CTL group (FIG. 3). CTL and Scl-Ab healing attachments appeared similar at 2 and 4 weeks of healing. However, after 8 weeks of healing, Scl-Ab treatment led to improved insertion continuity, integrity, and fiber alignment compared to CTL. Semi-quantitative, blinded analysis supported these observations, demonstrating a more mature tendon-to-bone attachment and new bone formation in the Scl-Ab group compared to CTL groups at 8 weeks (Table 3).

TABLE 3
The modified tendon-to-bone maturing score improved over time in all
groups with Scl-Ab treatment resulting in a lower total maturity score reflecting a more
mature attachment at 8 weeks of healing compared to control animals. Results are shown as
median (minimum, maximum).

| | 2 Week | | 4 Week | | 8 Week | |
|---|---|---|---|---|---|---|
| | CTL | Scl-Ab | CTL | Scl-Ab | CTL | Scl-Ab |
| Cellularity | 2 (1, 3) | 3 (2, 3) | 1 (1, 1) | 1 (1, 2) | 1 (1, 1) | 1 (1, 1) |
| Fibroblasts | 2 (1, 2) | 2 (2, 2) | 1 (1, 1) | 1.5 (1.5, 2) | 1 (1, 2) | 1 (1, 1) |
| Matrix | 4 (1, 4) | 2 (2, 3) | 3 (2.5, 3) | 2.5 (1, 3) | 2 (2, 3) | 1.5 (1, 2.5) |
| Cell Orientation | 3 (1, 3) | 2 (2, 3) | 2 (2, 3) | 1.5 (1, 3) | 2 (1, 2) | 1 (1, 1) |
| Collagen Orientation | 3 (1, 3) | 2 (2, 3) | 2 (2, 3) | 1.5 (1, 3) | 2 (1, 2) | 1 (1, 1) |
| Insertion Integrity | 3 (1, 4) | 1.5 (1.5, 2) | 2.5 (2.5, 3) | 2.5 (1, 2.5) | 2.5 (1.5, 3) | 1.5 (1, 2.5) |
| Insertion Continuity | 3 (1, 4) | 2 (2, 3) | 3 (3, 3) | 3 (1, 3) | 2 (1, 3) | 1 (1, 2) |
| Bone Resoprtion | 3 (2, 4) | 4 (3, 4) | 3 (2, 4) | 3 (3, 4) | 3 (3, 4) | 4 (4, 4) |
| Bone Modeling | 3 (1, 4) | 4 (3, 4) | 4 (3, 4) | 4 (3, 4) | 4 (4, 4) | 4 (4, 4) |
| Maturity | 24 (14, 29) | 22.5 (22, 23.5) | 21 (19.5, 25) | 20.5 (15.5, 24.5) | 21.5 (16.5, 22) | 16 (15, 19) |

Gene Expression in Mineralized Tissues (Bone and Fibrocartilage):

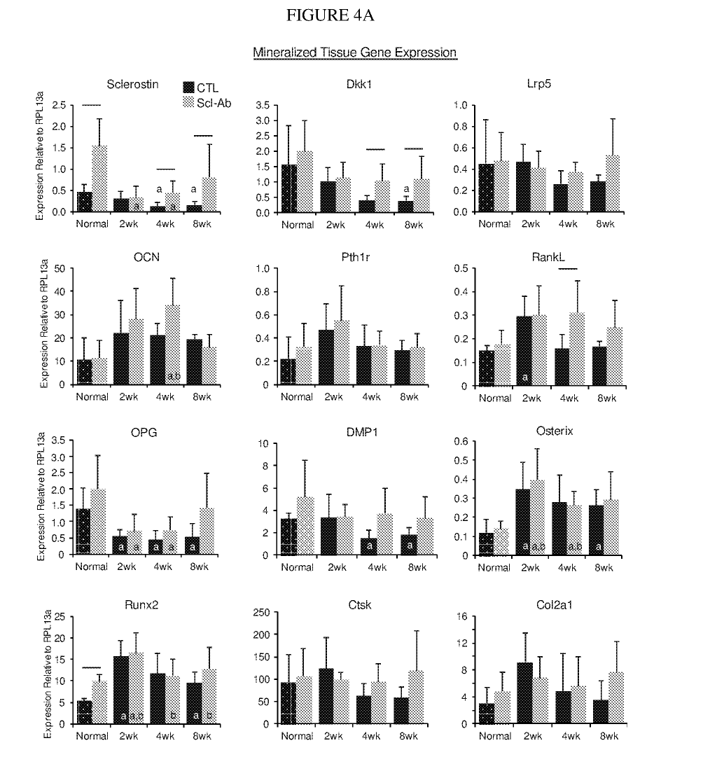

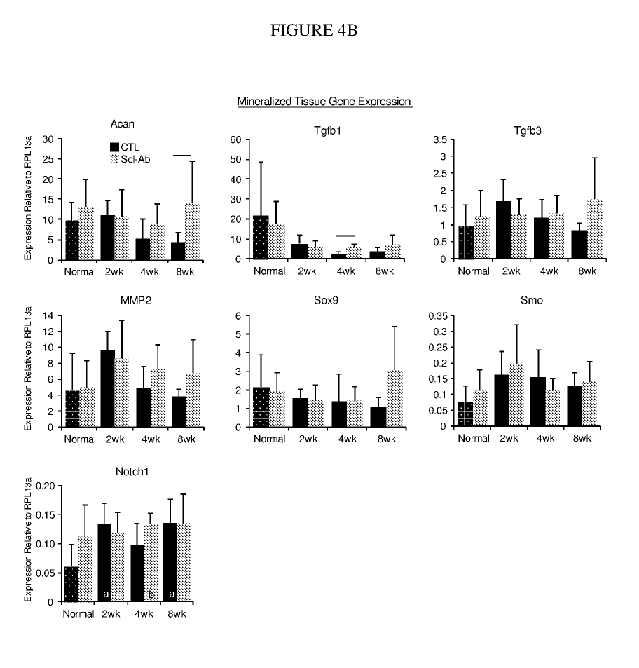

Scl-Ab treatment had a significant effect on expression of a number of genes in mineralized tissue near the tendon enthesis, including Sclerostin, Dkk1, RankL, DMP1, and Runx2 (FIG. 4). After 8 weeks of healing, expression of Sclerostin and Dkk1 were 3.3× and 2.5× greater in Scl-Ab compared to CTL, respectively. Expression of Lrp5 was not effected by treatment or healing time. Osteocalcin (OCN), a marker of osteoblast activity, was significantly increased following injury, while Osteoprotogerin (OPG), a marker of osteoclast inhibition, was significantly decreased after injury in all groups. Expression of RankL and DMP1 were both increased with Scl-Ab treatment (FIG. 4). There was no effect of Scl-Ab treatment on the fibrocartilage-related genes TGβ1, TGβ3, MMP2, Col2a1, and Sox9 (FIG. 4). Expression of Smo and Notch1, members of the hedgehog signaling pathway, was not affected by treatment.

Gene Expression in the Tendon:

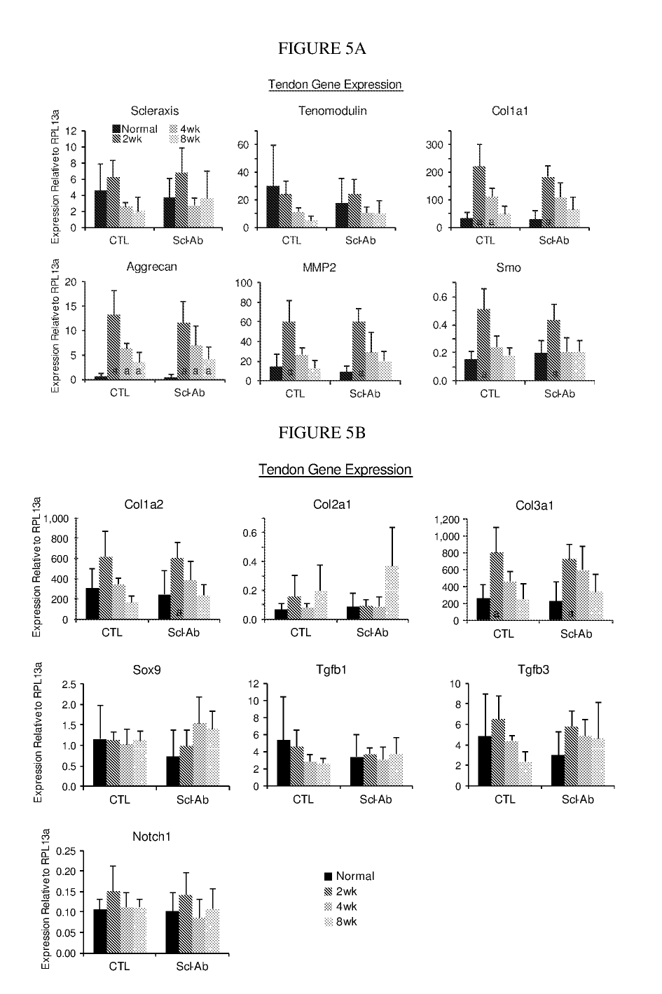

Scl-Ab treatment had no significant effect on tendon gene expression (FIG. 5). Healing time, however, significantly affected all tendon-related genes: Scleraxis, Tenomodoulin, Col1a1, Col1a2, Col3a1. Changes were most apparent at the 2 wk healing timepoint and trended towards normal by the 8 wk healing timepoint. Additionally, expression of Aggrecan was 20× greater in CTL and 17× greater with Scl-Ab treatment after 2 weeks of healing. Similarly, expression of Mmp2 was 4.1× greater in both CTL and Scl-Ab treatment groups after 2 weeks of healing.

Discussion:

Rotator cuff injury and repair lead to bone loss at the tendon-to-bone interface. A decrease in bone quantity and quality at the healing interface contributes to the high rates of re-tear following surgical repair [5]. The Example provided herein addressed bone loss during tendon-to-bone healing through sclerostin antibody treatment, by showing that enhancing the bony structure at the tendon attachment would lead to improved healing following rotator cuff injury and repair. Sclerostin antibody treatment increased indices of trabecular bone mass in the humeral head nearest to the healing tendon attachment. Although injury-associated bone loss remained in the control group after 8 weeks of healing, rapid recovery towards normal bone was seen by 8 weeks of healing in treated animals. The improvement in bone morphology at the healing interface had functional consequences, as demonstrated by improved attachment strength. Importantly, histological assessment further confirmed the benefit of sclerostin antibody treatment, with a more mature tendon-to-bone interface after 8 weeks of healing in the treated animals compared to control.

Delivery of bone anabolic agents like bone-morphogenetic protein 2 (BMP-2) to injury sites in an effort to improve bone mineral density and failure load following injury has been ineffective in canine and rodent model of tendon-to-bone repair [26, 28]. However, bisphosphonates have previously shown success in improving tendon-to-bone healing by reducing bone resorption [18, 29]. In a canine model of flexor tendon-to-bone healing, tendon injury caused bone mineral density near the tendon-to-bone interface to decrease by 29% compared to normal after 21 days [18]. An oral dose of alendronate was effective in preventing bone resorption leading to only a 6% decrease in bone mineral density compared to normal. The prevention of bone loss resulted in a significant improvement in the failure load of the repair after 21 days of healing. In a separate study, subcutaneous injections of zoledronic acid to ovariectomized rats resulted in a 23% increase in bone mineral density of the humeral head near the supraspinatus tendon insertion compared to control [29]. The increased bone mineral density was associated with a 24% increase in failure load at the interface following treatment, though no changes were observed in tendon histology score with bisphosphonate administration unlike with Scl-Ab. In the current study, after 8 weeks of healing, Scl-Ab treatment caused a 30% increase in bone mineral density and a 48% increase in failure load compared to control.

The sclerostin antibody used in the current study neutralizes sclerostin by preventing sclerostin binding to the Lrp5 receptor [30]. To determine whether improvements in bone morphology during tendon-to-bone healing were achieved through this mechanism, Wnt signaling-related gene expression was measured. Although there were no statistically significant changes in expression of Lrp5 due to Scl-Ab treatment, expression of sclerostin and Dkk1 were increased in treated bones during tendon-to-bone healing. This is in contrast to the decreased expression levels of these two genes in control bones compared to healthy bones, particularly at the later timepoints. The changes observed in the Scl-Ab group indicate a compensatory cellular response to the neutralized sclerostin, in a failed attempt to initiate Wnt signaling.

Further analysis of gene expression in the mineralized tissues at the healing attachment suggested increased osteoclast inhibition (as demonstrated by increased OPG expression) and an increase in osteoblast activity (as demonstrated by increased osteocalcin expression), consistent with the observed improvements in bone morphology. Following injury and repair, gene expression of osteoprogenitor markers (osterix and Runx2) were also significantly increased compared to normal in both treatment and control groups. Furthermore, Scl-Ab treatment led to increased Runx2 gene expression, in normal uninjured bone. Increased expression of these factors, which are also associated with differentiation of mesenchymal cells into osteoblasts [31, 32], indicates an overall induction of bone formation via progenitors as well as mature osteoblasts with Scl-Ab.

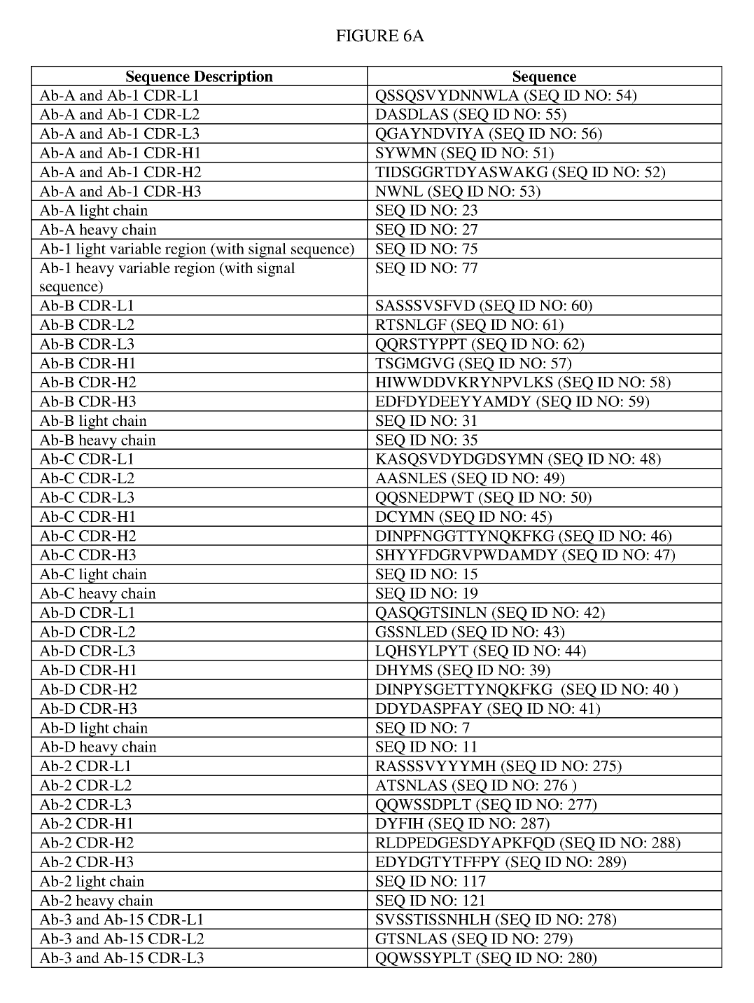

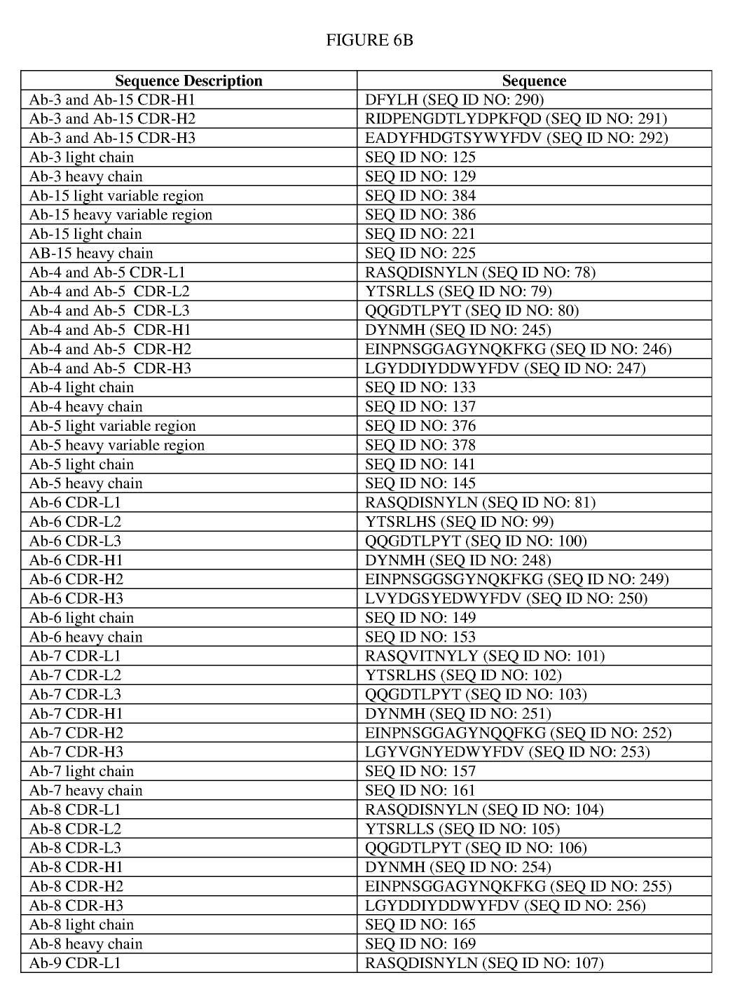

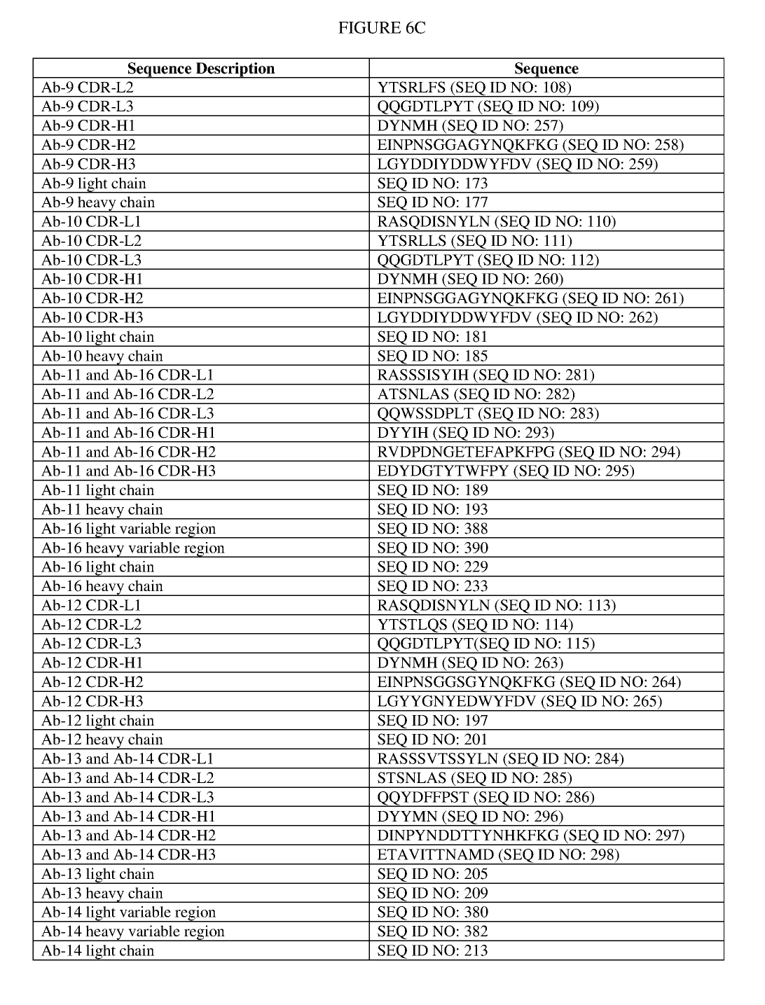

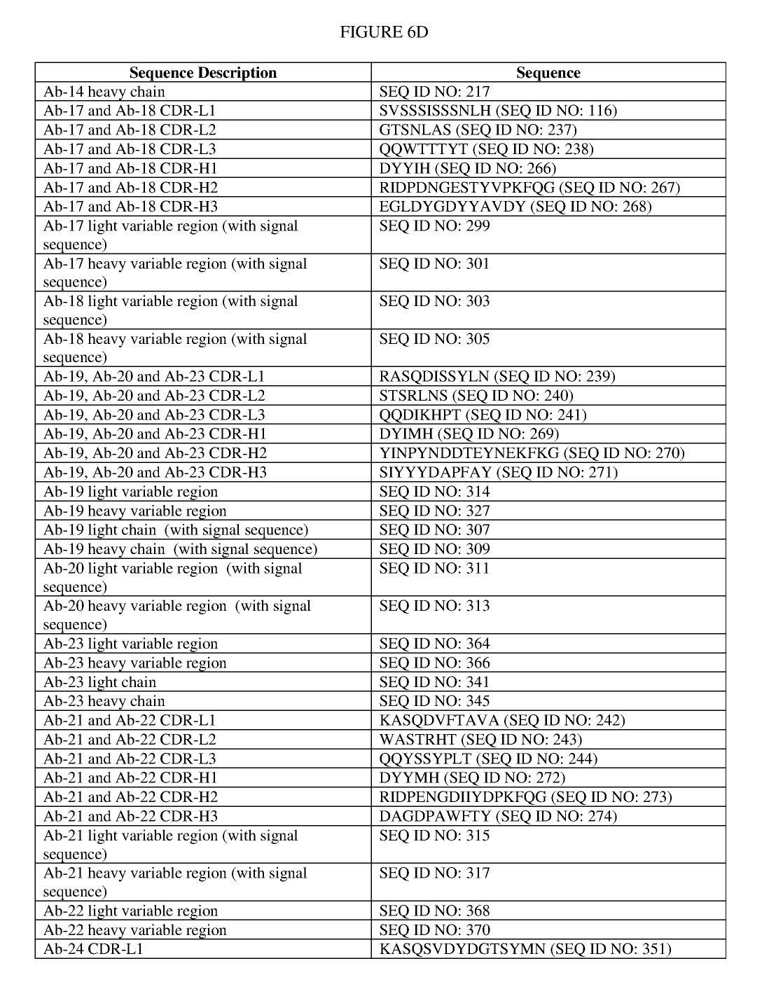

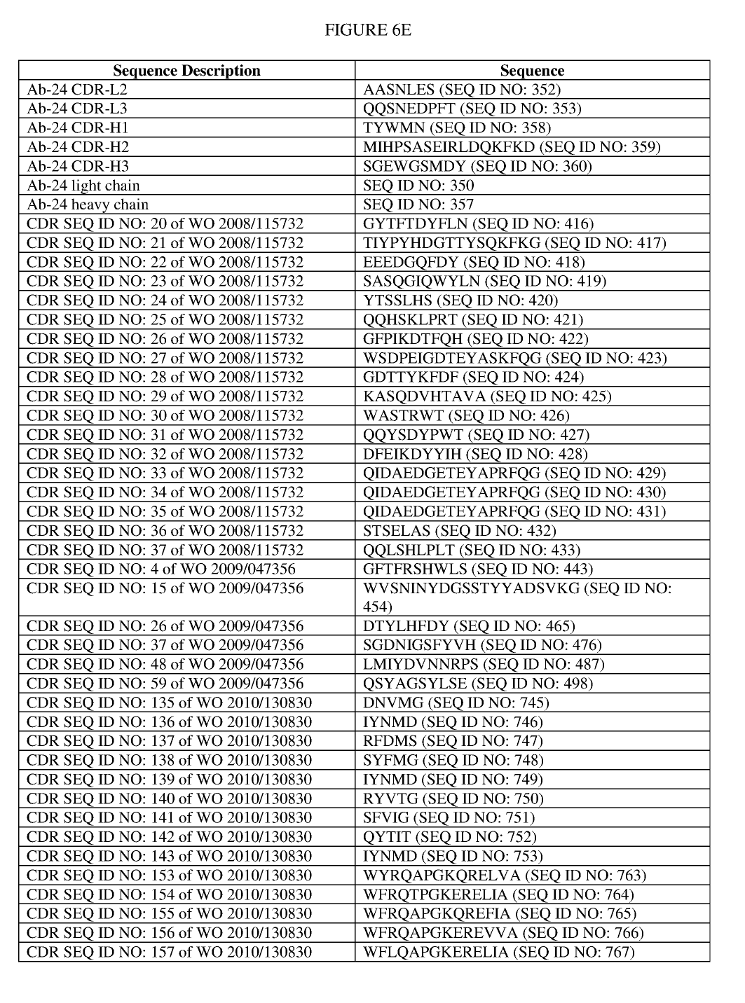

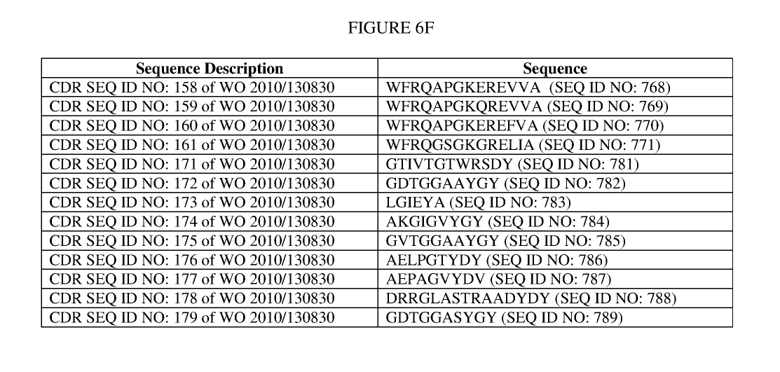

The strength of the tendon attachment is in large part dictated by the quality of the mineralized tissue at the interface [33, 34]. The healthy tendon-to-bone attachment has a gradient of mineral content across the fibrocartilaginous insertion and into the trabecular bone [35]. The increase in mechanical strength at the attachment due to Scl-Ab treatment is likely the result of improved mineralization in not only the adjacent trabecular bone (as measured by microCT), but also the fibrocartilage at the healing interface. Expression of aggrecan, an extracellular matrix marker or cartilage and fibrocartilage, was significantly higher in with Scl-Ab treatment in the mineralized tissue adjacent to the enthesis after 8 weeks of healing FIG. 6). Furthermore, semi-quantitative evaluation of histologic sections showed improvements in tendon-to-bone attachment maturity, including insertion integrity, after 4 and 8 weeks of healing with Scl-Ab treatment compared to control.

Scl-Ab treatment was applied systemically via subcutaneous injections. Therefore, all tissues, including the tendon adjacent to the healing interface as well as tissues in other joints also received Scl-Ab treatment. To evaluate possible effects of Scl-Ab on non-mineralized tissues, gene expression was examined in the supraspinatus tendon adjacent to the healing interface. Scl-Ab treatment did not have a significant effect on expression of genes in tendon tissue (FIG. 5), alleviating the concern of possible off-target tissue effects of treatment. However, Scl-Ab treatment did lead to a decrease in modulus and stiffness in healthy tendon-to-bone attachments (FIG. 2). This result is consistent with a previous finding that bisphosphonate treatment during tendon-to-bone healing can cause a decrease in stiffness [18]. Due to the built-in mechanical safety factor of tendons and ligaments for typical physiologic activities [36], the small decreases in stiffness and modulus should not predispose healthy tendons to injury.

In summary, the data provided herein demonstrates that treatment with a sclerostin antibody resulted in improved tendon-to-bone attachment compared to the control in the tested animal model. As expected, the sclerostin antibody augmented the trabecular bone region adjacent to the enthesis. Surprisingly, histological assessment showed that sclerostin antibody treatment promoted better integration of tendon and bone by 8 weeks of healing. Thus, treatment with a sclerostin antibody described herein can not only improve tendon-to-bone attachment by augmenting the bone mass in the adjacent insertion site, but also by improving the histology of the injured tendon. Furthermore, sclerostin antibody treatment can be considered for preventing unloading-related bone loss during the period prior to surgical repair of the torn tendon.

### REFERENCES

[1] Tashjian R Z. Epidemiology, Natural History, and Indications for Treatment of Rotator Cuff Tears. Clinics in Sports Medicine 2012; 31: 589-604.
[2] Lehman C, Cuomo F, Kummer F J, Zuckerman J D. The incidence of full thickness rotator cuff tears in a large cadaveric population. Bull Hosp Jt Dis 1995; 54: 30-1.
[3] Keener J D, Galatz L M, Teefey S A, Middleton W D, Steger-May K, Stobbs-Cucchi G, Patton R, Yamaguchi K. A Prospective Evaluation of Survivorship of Asymptomatic Degenerative Rotator Cuff Tears; 2015.
[4] Colvin A C, Egorova N, Harrison A K, Moskowitz A, Flatow E L. National trends in rotator cuff repair. J Bone Joint Surg Am 2012; 94: 227-33.
[5] Galatz L M, Ball C M, Teefey S A, Middleton W D, Yamaguchi K. The outcome and repair integrity of completely arthroscopically repaired large and massive rotator cuff tears. J Bone Joint Surg Am 2004; 86-A: 219-24.
[6] Thomopoulos S, Birman V, Genin G M. Structural interfaces and attachments in biology: Springer Science & Business Media; 2012.
[7] Galatz L M, Charlton N, Das R, Kim H M, Havlioglu N, Thomopoulos S. Complete removal of load is detrimental to rotator cuff healing. J Shoulder Elbow Surg 2009; 18: 669-75.
[8] Galatz L M, Rothermich S Y, Zaegel M, Silva M J, Havlioglu N, Thomopoulos S. Delayed repair of tendon to bone injuries leads to decreased biomechanical properties and bone loss. J Orthop Res 2005; 23: 1441-7.
[9] Gimbel J A, Van Kleunen J P, Lake S P, Williams G R, Soslowsky L J. The role of repair tension on tendon to bone healing in an animal model of chronic rotator cuff tears. Journal of Biomechanics 2007; 40: 561-568.
[10] Thomopoulos S, Kim H M, Rothermich S Y, Biederstadt C, Das R, Galatz L M. Decreased muscle loading delays maturation of the tendon enthesis during postnatal development. J Orthop Res 2007; 25: 1154-63.
[11] Thomopoulos S, Zampiakis E, Das R, Silva M J, Gelberman R H. The effect of muscle loading on flexor tendon-to-bone healing in a canine model. Journal of Orthopaedic Research 2008; 26: 1611-1617.
[12] Leppala J, Kannus P, Natri A, Pasanen M, Sievänen H, Vuori I, Järvinen M. Effect of anterior cruciate ligament injury of the knee on bone mineral density of the spine and affected lower extremity: a prospective one-year follow-up study. Calcified tissue international 1999; 64: 357-363.
[13] Alfredson H, Nordström P, Lorentzon R. Prolonged progressive calcaneal bone loss despite early weightbearing rehabilitation in patients surgically treated for Achilles tendinosis. Calcified tissue international 1998; 62: 166-171.
[14] Silva M J, Ritty T M, Ditsios K, Burns M E, Boyer M I, Gelberman R H. Tendon injury response: Assessment of biomechanical properties, tissue morphology and viability following flexor digitorum profundus tendon transection. Journal of Orthopaedic Research 2004; 22: 990-997.
[15] Killian M L, Cavinatto L, Shah S A, Sato E J, Ward S R, Havlioglu N, Galatz L M, Thomopoulos S. The effects of chronic unloading and gap formation on tendon-to-bone healing in a rat model of massive rotator cuff tears. Journal of Orthopaedic Research 2014; 32: 439-447.
[16] Killian M L, Cavinatto L M, Ward S R, Havlioglu N, Thomopoulos S, Galatz L M. Chronic Degeneration Leads to Poor Healing of Repaired Massive Rotator Cuff Tears in Rats. The American Journal of Sports Medicine 2015; 43: 2401-2410.
[17] Ditsios K, Boyer M I, Kusano N, Gelberman R H, Silva M J. Bone loss following tendon laceration, repair and passive mobilization. Journal of Orthopaedic Research 2003; 21: 990-996.
[18] Thomopoulos S, Matsuzaki H, Zaegel M, Gelberman R H, Silva M J. Alendronate prevents bone loss and improves tendon-to-bone repair strength in a canine model. Journal of Orthopaedic Research 2007; 25: 473-479.
[19] Kennel K A, Drake M T. Adverse Effects of Bisphosphonates: Implications for Osteoporosis Management. Mayo Clinic Proceedings 2009; 84: 632-638.
[20] Bone H G, Hosking D, Devogelaer J-P, Tucci J R, Emkey R D, Tonino R P, Rodriguez-Portales J A, Downs R W, Gupta J, Santora A C, Liberman U A. Ten Years' Experience with Alendronate for Osteoporosis in Postmenopausal Women. New England Journal of Medicine 2004; 350: 1189-1199.
[21] Black D M, Schwartz A V, Ensrud K E, et al. Effects of continuing or stopping alendronate after 5 years of treatment: The fracture intervention trial long-term extension (flex): a randomized trial. JAMA 2006; 296: 2927-2938.
[22] Li X, Ominsky M S, Warmington K S, Morony S, Gong J, Cao J, Gao Y, Shalhoub V, Tipton B, Haldankar R, Chen Q, Winters A, Boone T, Geng Z, Niu Q-T, Ke H Z, Kostenuik P J, Simonet W S, Lacey D L, Paszty C. Sclerostin Antibody Treatment Increases Bone Formation, Bone Mass, and Bone Strength in a Rat Model of Postmenopausal Osteoporosis. Journal of Bone and Mineral Research 2009; 24: 578-588.
[23] McClung M R, Grauer A, Boonen S, Bolognese M A, Brown J P, Diez-Perez A, Langdahl B L, Reginster J-Y, Zanchetta J R, Wasserman S M, Katz L, Maddox J, Yang Y-C, Libanati C, Bone H G. Romosozumab in Postmenopausal Women with Low Bone Mineral Density. New England Journal of Medicine 2014; 370: 412-420.
[24] Padhi D, Jang G, Stouch B, Fang L, Posvar E. Single-dose, placebo-controlled, randomized study of AMG 785, a sclerostin monoclonal antibody. Journal of Bone and Mineral Research 2011; 26: 19-26.
[25] Manning C N, Kim H M, Sakiyama-Elbert S, Galatz L M, Havlioglu N, Thomopoulos S. Sustained delivery of transforming growth factor beta three enhances tendon-to-bone healing in a rat model. J Orthop Res 2011; 29: 1099-105.
[26] Lipner J, Shen H, Cavinatto L, Liu W, Havlioglu N, Xia Y, Galatz L M, Thomopoulos S. In Vivo Evaluation of Adipose-Derived Stromal Cells Delivered with a Nanofiber Scaffold for Tendon-to-Bone Repair. Tissue Engineering Part A 2015; 21: 2766-2774.
[27] Ide J, Kikukawa K, Hirose J, Iyama K-i, Sakamoto H, Fujimoto T, Mizuta H. The effect of a local application of fibroblast growth factor-2 on tendon-to-bone remodeling in rats with acute injury and repair of the supraspinatus tendon. Journal of Shoulder and Elbow Surgery 2009; 18: 391-398.
[28] Thomopoulos S, Kim H M, Silva M J, Ntouvali E, Manning C N, Potter R, Seeherman H, Gelberman R H. THE EFFECT OF BONE MORPHOGENETIC PROTEIN 2 ON TENDON-TO-BONE HEALING IN A CANINE FLEXOR TENDON MODEL. Journal of orthopaedic research: official publication of the Orthopaedic Research Society 2012; 30: 1702-1709.
[29] Cadet E R, Vorys G C, Rahman R K, Park S-H, Gardner T R, Lee F Y, Levine W N, Bigliani L U, Ahmad C S. Improving bone density at the rotator cuff footprint increases supraspinatus tendon failure stress in a rat model. Journal of Orthopaedic Research 2010; 28: 308-314.
[30] Sinder B P, Salemi J D, Ominsky M S, Caird M S, Marini J C, Kozloff K M. Rapidly growing Brtl/+ mouse model of osteogenesis imperfecta improves bone mass and strength with sclerostin antibody treatment. Bone 2015; 71: 115-123.
[31] Komori T. Regulation of osteoblast differentiation by transcription factors. Journal of Cellular Biochemistry 2006; 99: 1233-1239.
[32] Nakashima K, Zhou X, Kunkel G, Zhang Z, Deng J M, Behringer R R, de Crombrugghe B. The Novel Zinc Finger-Containing Transcription Factor Osterix Is Required for Osteoblast Differentiation and Bone Formation. Cell 2002; 108: 17-29.
[33] Genin G M, Kent A, Birman V, Wopenka B, Pasteris J D, Marquez P J, Thomopoulos S. Functional grading of mineral and collagen in the attachment of tendon to bone. Biophys J 2009; 97: 976-85.
[34] Schwartz A G, Lipner J H, Pasteris J D, Genin G M, Thomopoulos S. Muscle loading is necessary for the formation of a functional tendon enthesis. Bone 2013; 55: 44-51.
[35] Schwartz A G, Pasteris J D, Genin G M, Daulton T L, Thomopoulos S. Mineral distributions at the developing tendon enthesis. PLoS One 2012; 7: e48630.
[36] West J R, Juncosa N, Galloway M T, Boivin G P, Butler D L. Characterization of in vivo Achilles tendon forces in rabbits during treadmill locomotion at varying speeds and inclinations. J Biomech 2004; 37: 1647-53.
[37] Sharma P, Maffulli N. Tendon Injury and Tendinopathy: Healing and Repair; 2005.

What is claimed is:

1. A method for enhancing connective tissue-to-bone healing in a subject in need thereof comprising administering to the subject an anti-sclerostin antibody in an amount effective to enhance connective tissue-to-bone healing in the subject.

2. The method of claim 1, wherein the connective tissue is a ligament, tendon, meniscus or a labrum.

3. The method of claim 1, wherein the connective tissue is a tendon.

4. The method of any one of claims 1-3, wherein the anti-sclerostin antibody is administered in an amount from about 90-270 mg.

5. The method of any one of claims 1-4, wherein the anti-sclerostin antibody is administered systemically.

6. The method of any one of claims 1-4, wherein the anti-sclerostin antibody is incorporated into a gel, a sponge, or matrix and implanted locally.

7. The method of any one of claims 1-3, wherein the anti-sclerostin antibody is an immunoglobulin comprising a heavy chain and a light chain.

8. The method of any one of claims 1-7, wherein the anti-sclerostin antibody is an antibody or fragment thereof that demonstrates a binding affinity for sclerostin of SEQ ID NO: 1 of less than or equal to 1×10 M.

9. The method of any one of claims 1-8, where the anti-sclerostin antibody cross-blocks the binding of at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24 to sclerostin and/or is cross-blocked from binding to sclerostin by at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24.

10. The method of any one of claims 1-8, wherein the anti-sclerostin antibody comprises a CDR-H1 of SEQ ID NO:245, a CDR-H2 of SEQ ID NO:246, a CDR-H3 of SEQ ID NO:247, a CDR-L1 of SEQ ID NO:78, a CDR-L2 of SEQ ID NO:79, and a CDR-L3 of SEQ ID NO:80.

11. The method of claim 10, wherein the anti-sclerostin antibody comprises heavy chains comprising SEQ ID NO: 378 and light chains comprising SEQ ID NO 376.

12. The method of any one of claims 1-10, wherein the anti-sclerostin antibody is formulated into a pharmaceutical composition comprising 55 mM acetate, 13 mm calcium, 6.0% (w/v) sucrose, 0.006% (w/v) polysorbate 20, at pH 5.2.

13. The method of claim 12, wherein the pharmaceutical composition comprises 90 mg/mL anti-sclerostin antibody.

14. A method of improving the outcome of a connective tissue reattachment procedure in a subject in need thereof comprising administering to the subject an anti-sclerostin antibody in an amount effective to improve the outcome of procedure.

15. The method of claim 4, wherein the procedure is rotator cuff repair, Achilles tendon repair, patellar-patella tendon repair, medial cruciate ligament (MCL) reconstruction, anterior cruciate ligament (ACL) reconstruction, ulnar collateral ligament (UCL), meniscus repair, or labrum repair.

16. The method of claim 14, wherein the procedure comprises graft attachment, and the anti-sclerostin antibody is applied to the graft ex vivo.

17. The method of any one of claims 14-16 wherein the connective tissue is a ligament, tendon, meniscus or labrum.

18. The method of any one of claims 14-16, wherein the connective tissue is a tendon.

19. The method of any one of claims 14-18, wherein the anti-sclerostin antibody is administered in an amount from about 90 mg-270 mg.

20. The method of any one of claims 14-19, wherein the anti-sclerostin antibody is administered systemically.

21. The method of any one of claims 14-19, wherein the anti-sclerostin antibody is incorporated into a gel, a sponge, or matrix and implanted locally.

22. The method of any one of claims 14-21, wherein the anti-sclerostin antibody is an immunoglobulin comprising a heavy chain and a light chain.

23. The method of any one of claims 14-22, wherein the anti-sclerostin antibody is an antibody or fragment thereof that demonstrates a binding affinity for sclerostin of SEQ ID NO: 1 of less than or equal to 1×10⁻⁹ M.

24. The method of any one of claims 14-23, where the anti-sclerostin antibody cross-blocks the binding of at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24 to sclerostin and/or is cross-blocked from binding to sclerostin by at least one of antibodies Ab-A, Ab-B, Ab-C, Ab-D, Ab-1, Ab-2, Ab-3, Ab-4, Ab-5, Ab-6, Ab-7, Ab-8, Ab-9, Ab-10, Ab-11, Ab-12, Ab-13, Ab-14, Ab-15, Ab-16, Ab-17, Ab-18, Ab-19, Ab-20, Ab-21, Ab-22, Ab-23, and Ab-24.

24. The method of any one of claims 14-23, wherein the anti-sclerostin antibody comprises a CDR-H1 of SEQ ID NO:245, a CDR-H2 of SEQ ID NO:246, a CDR-H3 of SEQ ID NO:247, a CDR-L1 of SEQ ID NO:78, a CDR-L2 of SEQ ID NO:79, and a CDR-L3 of SEQ ID NO:80.

25. The method of claim 24, wherein the anti-sclerostin antibody comprises heavy chains comprising SEQ ID NO: 378 and light chains comprising SEQ ID NO 376.

26. The method of any one of claims 14-25, wherein the anti-sclerostin antibody is formulated into a pharmaceutical composition comprising 55 mM acetate, 13 mm calcium, 6.0% (w/v) sucrose, 0.006% (w/v) polysorbate 20, at pH 5.2.

27. The method of claim 26, wherein the pharmaceutical composition comprises 90 mg/mL anti-sclerostin antibody.
